(19) United States
(12) Patent Application Publication
(10) Pub. No.: US 2013/0089627 A1
(43) Pub. Date: Apr. 11, 2013

(54) METHOD FOR TREATING A CANCER CAUSED BY CANCER STEM CELLS

(71) Applicant: NEW BELLUS ENTERPRISES CO., LTD., Tainan City (TW)

(72) Inventors: Chun-Chin HUANG, Tainan City (TW); Chih Chieh CHEN, Tainan City (TW); Lih-Geeng CHEN, Tainan City (TW)

(73) Assignee: NEW BELLUS ENTERPRISES CO., LTD., Tainan City (TW)

(21) Appl. No.: 13/649,984

(22) Filed: Oct. 11, 2012

(30) Foreign Application Priority Data
Oct. 11, 2011 (TW) 100136825

(51) Int. Cl. A61K 31/22; A61N 5/10; A61P 35/00; A61P 35/02; A61K 31/337; A61K 33/24

(52) U.S. Cl. 424/649; 514/546; 514/449; 600/1

(57) ABSTRACT

This invention provides a method for treating a cancer caused by cancer stem cells in a subject in need thereof, comprising administering to the subject an effective amount of Antrodia camphorata extracts. The invention also provides a method for treating a cancer caused by cancer stem cells in a subject in need thereof, comprising administering to the subject an effective amount of 4-acetyl-antroquinonol B.

### FIELD OF THE INVENTION

The present invention is related to a method for treating cancer, comprising administering an effective amount of Antrodia camphorata extracts to the subject.

### BACKGROUND OF THE INVENTION

### Cancer Stem Cells

The traditional cancer treatment is mainly to inhibit the growth of the fast proliferating cancer cells and to induce their apoptosis. However, because of the heterogeneity of cancer cells, cancer cells with high grades of malignancy often survive or escape from the detection of the immune system that leads to frequent recurrence of cancer after treatments of chemotherapy drugs and radiation therapy. In recent years, the rise of a new theory provides a new explanation for the reason why cancers are difficult to cure. Many studies indicate that the majority of cancer cells do not have the ability to cause tumor, only a very small portion of cancer cells are tumorigenic and can differentiate into a variety of cells in tumor tissue. Scientists found that these few cells in different cancer tissues, including leukemia, breast cancer, brain cancer, ovarian cancer, prostate cancer, colorectal cancer and oral cancer, have more resistance to radiation or drugs than other cancer cells. Therefore, these cancer cells with stem cell-like characteristics are named “cancer stem cells” (CSCs).

Current studies have indicated that cancer stem cells can be isolated from patients' tumor tissues. A handful of “cancer stem cells” can form a tumor in the patient's body. The regulation of activating and proliferating of such cancer stem cells is closely related to tumor recurrence, remote invasion and even the patient's survival rate. Similar to the normal stem cells, cancer stem cells also have the ability to self-renew and differentiate. They grow continuously and differentiate into tumor cells of different types and morphologies. However, unlike normal stem cells, the self-renewal mechanisms of cancer stem cells are not under normal regulation. Take the normal stem cell surface antigen CD133 for an example, CD133 is a glycoprotein having five transmembrane domains that was first identified from CD34⁺ precursor cells isolated from blood of adult, bone marrow and fetal liver cells and was regarded as a marker of hematopoietic stem cells. However, in the study of last five years, CD133 is regarded as a surface marker for the cancer stem cell of leukemia, brain cancer, retinoblastoma, kidney cancer, pancreatic cancer, prostate cancer and liver cancer. Recent reports also pointed out that there may be CD133⁺ cancer stem cells in medulloblastoma as well as glioma and the ability of proliferating and self-renewal of these CD133⁺ cells are better than the general tumor cells. Therefore, CD133 can be regarded as one of the important markers of cancer stem cells.

### The Discrimination of Cancer Stem Cells

According to the characteristics of cancer stem cells, three ways are provided to successfully isolate cancer stem cells from solid tumors. First, based on the specific surface antigen expressed on the cancer stem cells, such as CD44 or of CD133, flow cytometry was used to isolate the cancer stem cells. CD133 was isolated from the cancer stem cells of a variety of brain tumors, including glioblastoma multiforme, children medulloblastoma and ependymomas. In addition, it was also found in the cancer stem cells of colon cancer. There are approximately 1.8-24.5% of cells expressing CD133 in colon cancer and most of these cells have the ability to form tumors. Second, the fluorescent dye Hoechst 33342 was used to stain tumor tissues or cancer cell groups and then the side population without fluorescent signals, which are cancer stem cells, was isolated. These cells may lead to tumor chemoresistance. The expression of ABCG2, an ATPase transporter, is closely related to the side population phenomenon. Because of the high expression of ABCG2 on cell membrane of stem cells, the transporter will actively pump Hoechst 33342 from inside of the nucleus to outside. These side populations of cells analyzed by flow cytometry were defined as cells with the characteristic of stem cells. However, the latest studies have shown that the cancer cells have similar tumorigenicity regardless of their expression of ABCG2. Third, the tumor tissue or cancer cells were cultured in medium that is serum-free but containing specific growth factors, such as basic fibroblast growth factor (bFGF), epidermal growth factor (EGF) and other synthetic growth factors. The sphere body formation cells are abundant in cancer stem cells. It is assumed that the serum-free culture environment can help the cancer stem cells maintain in the undifferentiated state.

Molecular markers are mainly used to confirm the cell surface antigen or specific transcription factors. Various types of stem cells and cancer stem cells need to be confirmed by using different markers and then detecting the ability of self-renewal and differentiation of the stem cells. Therefore, the primary goal of the top research teams in various countries is to search for the surface markers or specific gene cluster unique and specific to cancer stem cells and identify the cancer stem cells with tumorigenic ability. In addition, if the cancer stem cells can be correctly and successfully isolated, the basic research of the subsequent gene regulation, human body repair or the development of drug screening platform or the direct application of individualized anti-cancer treatment in cancer patients can be conducted by in vitro culture.

### Chemoresistance & Radioresistance

Recently, the kinds of cancer stem cells are regarded as cells having the potential to develop into cancers. In the experimental animal model, it is also proved that a very small amount of cancer stem cells is enough to form tumors. However, other non cancer stem cells need much greater number of cells to achieve similar tumorigenicity. On the other hand, because of the proliferating rate of the cancer stem cells are extremely slow, even in the non-dividing state, and the expressing amount of ABC transport proteins is far more than the normal cancer cells, the cancer stem cells cannot be killed easily by chemotherapy drugs or radiation. Thus, the cancer stem cells are not only the main reason for cancer recurrence after treatment and the ineffectiveness of drugs but also the main reason for malignant cancer metastasis. This shows that the opportunity to cure cancers is to eliminate the cancer stem cells. Therefore, one of the important topics in recent cancer research is to identify the difference between such cells and the normal cancer cells or normal stem cells, to develop effective strategy for killing cancer stem cells specific to such features.

In recent years, the medical profession proposes a new vision for such difficult-to-cure and easy-to-relapse and metastasis situation. The specific “cancer stem cell” may exist in different cancer tissues. It is the key point to cause tumor recurrence in cancer patients. The traditional cancer treatments are radiation and chemotherapy after surgery to inhibit the growth of cancer cells and to induce their apoptosis. However, the cancer cells with high malignancy can survive from chemotherapy drugs and radiation treatment and escape from the detection of the immune system that are easily recurrent after treatments.

### Antrodia Camphorate

Antrodia camphorata is also called Niu Chang-Zhi, Niu Chang-Gu, red camphor mushroom and the like, which is a perennial mushroom belonging to the order Aphyllophorales, the family Polyporaceae. It is an endemic species in Taiwan growing on the inner rotten heart wood wall of Cinnamomum kanehirae Hay. Cinnamoum kanehirai Hay is rarely distributed and being overcut unlawfully, which makes Antrodia camphorata growing inside the tree in the wild became even rare. The price of Antrodia camphorata is very expensive due to the extremely slow growth rate of natural Antrodia camphorata that only grows between June to October.

In traditional Taiwanese medicine, Antrodia camphorata is commonly used as an antidotal, liver protective, anti-cancer drug. Antrodia camphorata, like general edible and medicinal mushrooms, is rich in numerous nutrients including triterpenoids, polysaccharides (such as [beta]-glucosan), adenosine, vitamins (such as vitamin B, nicotinic acid), proteins (immunoglobulins), superoxide dismutase (SOD), trace elements (such as calcium, phosphorus and germanium and so on), nucleic acid, steroids, and stabilizers for blood pressure (such as antodia acid) and the like. These physiologically active ingredients are believed to exhibit effects such as: anti-tumor activities, increasing immuno-modulating activities, anti-allergy, anti-bacteria, anti-high blood pressure, decreasing blood sugar, decreasing cholesterol, and the like. Now there are only researches for the inhibitory effects of Antrodia camphorata for normal cancer cells, but no researches for the cancer stem cells.

### BRIEF DESCRIPTION OF THE DRAWINGS

FIG. 1 shows the producing process of A. camphorata extracts used in the present invention.

FIG. 2 shows the ESI(+)-MS Mass spectrum of 4-acetyl-antroquinonol B.

FIG. 3 shows the UV absorption spectra of 4-acetyl-antroquinonol B.

FIG. 4 shows the ¹H-NMR (500 MHz, CDCl₃) spectra of 4-acetyl-antroquinonol B.

FIG. 5 shows the ¹³C-NMR (125 MHz, CDCl₃) spectra of 4-acetyl-antroquinonol B.

FIG. 6 shows the pH of A. camphorata concentrates and the figures for inhibiting the growth of human stem cells.

FIG. 7 shows the cytotoxicity curve of A. camphorata concentrate (D1) for inhibiting human Lung cancer stem cell (Lung CSC).

FIG. 8 shows the cytotoxicity curve of ethyl acetate extracts of A. camphorata concentrate (D2) for inhibiting human Lung cancer stem cell (Lung CSC).

FIG. 9 shows the cytotoxicity curve of lyophilized powder of A. camphorata concentrate (D3) for inhibiting human Lung cancer stem cell (Lung CSC).

FIG. 10 shows the cytotoxicity curve of ethyl acetate extracts of lyophilized powder of A. camphorata concentrate (D4) for inhibiting human Lung cancer stem cell (Lung CSC).

FIG. 11 shows the cytotoxicity curve of ethyl acetate extracts of A. camphorata mycelium (D5) for inhibiting human Lung cancer stem cell (Lung CSC).

FIG. 12 shows the cytotoxicity curve of A. camphorata concentrate (D1) for inhibiting human fibroblast AF-1 (Adult fibroblast-1).

FIG. 13 shows the cytotoxicity curve of ethyl acetate extracts of A. camphorata concentrate (D2) for inhibiting human fibroblast AF-1 (Adult fibroblast-1).

FIG. 14 shows the cytotoxicity curve of lyophilized powder of A. camphorata concentrate (D3) for inhibiting human fibroblast AF-1 (Adult fibroblast-1).

FIG. 15 shows the cytotoxicity curve of ethyl acetate extracts of lyophilized powder of A. camphorata concentrate (D4) for inhibiting human fibroblast AF-1 (Adult fibroblast-1).

FIG. 16 shows the cytotoxicity curve of ethyl acetate extracts of A. camphorata mycelium (D5) for inhibiting human fibroblast AF-1 (Adult fibroblast-1).

FIG. 17 shows the cytotoxicity curve of A. camphorata concentrate (D1) for inhibiting Glioblastomas multiform cancer stem cells (GBM CSC).

FIG. 18 shows the cytotoxicity curve of ethyl acetate extracts of A. camphorata concentrate (D2) for inhibiting Glioblastomas multiform cancer stem cells (GBM CSC).

FIG. 19 shows the cytotoxicity curve of lyophilized powder of A. camphorata concentrate (D3) for inhibiting Glioblastomas multiform cancer stem cells (GBM CSC).

FIG. 20 shows the cytotoxicity curve of ethyl acetate extracts of lyophilized powder of A. camphorata concentrate (D4) for inhibiting Glioblastomas multiform cancer stem cells (GBM CSC).

FIG. 21 shows the cytotoxicity curve of ethyl acetate extracts of A. camphorata mycelium (D5) for inhibiting Glioblastomas multiform cancer stem cells (GBM CSC).

FIG. 22 shows the cytotoxicity curve of A. camphorata concentrate (D1) for inhibiting human fibroblast AF-2 (Adult fibroblast-2).

FIG. 23 shows the cytotoxicity curve of ethyl acetate extracts of A. camphorata concentrate (D2) for inhibiting human fibroblast AF-2 (Adult fibroblast-2).

FIG. 24 shows the cytotoxicity curve of lyophilized powder of A. camphorata concentrate (D3) for inhibiting human fibroblast AF-2 (Adult fibroblast-2).

FIG. 25 shows the cytotoxicity curve of ethyl acetate extracts of lyophilized powder of A. camphorata concentrate (D4) for inhibiting human fibroblast AF-2 (Adult fibroblast-2).

FIG. 26 shows the cytotoxicity curve of ethyl acetate extracts of A. camphorata mycelium (D5) for inhibiting human fibroblast AF-2 (Adult fibroblast-2).

FIG. 27 shows the cytotoxicity curve of A. camphorata concentrate (D1) for inhibiting Head and neck squamous cell carcinoma cancer stem cells (HNSCC CSC).

FIG. 28 shows the cytotoxicity curve of ethyl acetate extracts of A. camphorata concentrate (D2) for inhibiting Head and neck squamous cell carcinoma cancer stem cells (HNSCC CSC).

FIG. 29 shows the cytotoxicity curve of lyophilized powder of A. camphorata concentrate (D3) for inhibiting Head and neck squamous cell carcinoma cancer stem cells (HNSCC CSC).

FIG. 30 shows the cytotoxicity curve of ethyl acetate extracts of lyophilized powder of A. camphorata concentrate (D4) for inhibiting Head and neck squamous cell carcinoma cancer stem cells (HNSCC CSC).

FIG. 31 shows the cytotoxicity curve of ethyl acetate extracts of A. camphorata mycelium (D5) for inhibiting Head and neck squamous cell carcinoma cancer stem cells (HNSCC CSC).

FIG. 32 shows the cytotoxicity curve of A. camphorata concentrate (D1) for inhibiting colorectal cancer stem cells (CRC CSC).

FIG. 33 shows the cytotoxicity curve of ethyl acetate extracts of A. camphorata concentrate (D2) for inhibiting colorectal cancer stem cells (CRC CSC).

FIG. 34 shows the cytotoxicity curve of lyophilized powder of A. camphorata concentrate (D3) for inhibiting colorectal cancer stem cells (CRC CSC).

FIG. 35 shows the cytotoxicity curve of ethyl acetate extracts of lyophilized powder of A. camphorata concentrate (D4) for inhibiting colorectal cancer stem cells (CRC CSC).

FIG. 36 shows the cytotoxicity curve of ethyl acetate extracts of A. camphorata mycelium (D5) for inhibiting colorectal cancer stem cells (CRC CSC).

FIG. 37 shows the cytotoxicity curve of A. camphorata concentrate (D1) for inhibiting breast cancer stem cells (Breast CSC).

FIG. 38 shows the cytotoxicity curve of ethyl acetate extracts of A. camphorata concentrate (D2) for inhibiting breast cancer stem cells (Breast CSC).

FIG. 39 shows the cytotoxicity curve of lyophilized powder of A. camphorata concentrate (D3) for inhibiting breast cancer stem cells (Breast CSC).

FIG. 40 shows the cytotoxicity curve of ethyl acetate extracts of lyophilized powder of A. camphorata concentrate (D4) for inhibiting breast cancer stem cells (Breast CSC).

FIG. 41 shows the cytotoxicity curve of ethyl acetate extracts of A. camphorata mycelium (D5) for inhibiting breast cancer stem cells (Breast CSC).

FIG. 42 shows the cytotoxicity curve of ethyl acetate extracts of lyophilized powder of A. camphorata concentrate (D4) for inhibiting hepatoma cancer stem cells (Hepatoma CSC).

FIG. 43 shows the cytotoxicity curve of ethyl acetate extracts of A. camphorata mycelium for inhibiting hepatoma cancer stem cells (Hepatoma CSC).

FIG. 44 shows the cytotoxicity curve of ethyl acetate extracts of lyophilized powder of A. camphorata concentrate (D4) for inhibiting leukemia cancer stem cells (Leukemia CSC).

FIG. 45 shows the cytotoxicity curve of ethyl acetate extracts of A. camphorata mycelium (D5) for inhibiting leukemia cancer stem cells (Leukemia CSC).

FIG. 46 shows the cytotoxicity curve of ethyl acetate extracts of lyophilized powder of A. camphorata concentrate (D4) for inhibiting gastric cancer stem cells (Gastric CSC).

FIG. 47 shows the cytotoxicity curve of ethyl acetate extracts of A. camphorata mycelium (D5) for inhibiting gastric cancer stem cells (Gastric CSC).

FIG. 48 shows the effect of the combination of Ionizing Radiation treatment (2 Gy) for Glioblastomas multiform cancer stem cells (GBM CSC).

FIG. 49 shows the effect of the combination of Ionizing Radiation treatment (2 Gy) for lung cancer stem cells (Lung CSC).

FIG. 50 shows the effect of the combination of Ionizing Radiation treatment (2 Gy) for Head and neck squamous cell carcinoma cancer stem cells (HNSCC CSC).

FIG. 51 shows the effect of the combination of Ionizing Radiation treatment (2 Gy) for breast cancer stem cells (Breast CSC).

FIG. 52 shows the effect of the combination of Ionizing Radiation treatment (2 Gy) for hepatoma cancer stem cells (Hepatoma CSC).

FIG. 53 shows the effect of the combination of Ionizing Radiation treatment (2 Gy) for colorectal cancer stem cells (CRC CSC).

FIG. 54 shows the effect of the combination of Ionizing Radiation treatment (4 Gy) for Glioblastomas multiform cancer stem cells (GBM CSC).

FIG. 55 shows the effect of the combination of Ionizing Radiation treatment (4 Gy) for lung cancer stem cells (Lung CSC).

FIG. 56 shows the effect of the combination of Ionizing Radiation treatment (4 Gy) for Head and neck squamous cell carcinoma cancer stem cells (HNSCC CSC).

FIG. 57 shows the effect of the combination of Ionizing Radiation treatment (4 Gy) for breast cancer stem cells (Breast CSC).

FIG. 58 shows the effect of the combination of Ionizing Radiation treatment (4 Gy) for hepatoma cancer stem cells (Hepatoma CSC).

FIG. 59 shows the effect of the combination of Ionizing Radiation treatment (4 Gy) for colorectal cancer stem cells (CRC CSC).

FIG. 60 shows the effect of the combination of chemotherapy drugs treatment (Cisplatin, 10 μg/ml) for Glioblastomas multiform cancer stem cells (GBM CSC).

FIG. 61 shows the effect of the combination of chemotherapy drugs treatment (Cisplatin, 10 μg/ml) for lung cancer stem cells (Lung CSC).

FIG. 62 shows the effect of the combination of chemotherapy drugs treatment (Cisplatin, 10 μg/ml) for Head and neck squamous cell carcinoma cancer stem cells (HNSCC CSC).

FIG. 63 shows the effect of the combination of chemotherapy drugs treatment (Cisplatin, 10 μg/ml) for breast cancer stem cells (Breast CSC).

FIG. 64 shows the effect of the combination of chemotherapy drugs treatment (Cisplatin, 10 μg/ml) for hepatoma cancer stem cells (Hepatoma CSC).

FIG. 65 shows the effect of the combination of chemotherapy drugs treatment (Cisplatin, 10 μg/ml) for colorectal cancer stem cells (CRC CSC).

FIG. 66 shows the effect of the combination of chemotherapy drugs treatment (Taxol, 5 ng/ml) for Glioblastomas multiform cancer stem cells (GBM CSC).

FIG. 67 shows the effect of the combination of chemotherapy drugs treatment (Taxol, 5 ng/ml) for lung cancer stem cells (Lung CSC).

FIG. 68 shows the effect of the combination of chemotherapy drugs treatment (Taxol, 5 ng/ml) for Head and neck squamous cell carcinoma cancer stem cells (HNSCC CSC).

FIG. 69 shows the effect of the combination of chemotherapy drugs treatment (Taxol, 5 ng/ml) for breast cancer stem cells (Breast CSC).

FIG. 70 shows the effect of the combination of chemotherapy drugs treatment (Taxol, 5 ng/ml) for hepatoma cancer stem cells (Hepatoma CSC).

FIG. 71 shows the effect of the combination of chemotherapy drugs treatment (Taxol, 5 ng/ml) for colorectal cancer stem cells (CRC CSC).

FIG. 72 shows the cytotoxicity curve of 4-acetyl-antroquinonol B for inhibiting Lung adenocarcinoma CD133 positive cancer stem cells.

FIG. 73 shows the cytotoxicity curve of 4-acetyl-antroquinonol B for inhibiting oral cancer stem cells (Oral CSC).

FIG. 74 shows the cytotoxicity curve of 4-acetyl-antroquinonol B for inhibiting Glioblastomas multiform cancer stem cells (GBM CSC).

FIG. 75 shows the cytotoxicity curve of 4-acetyl-antroquinonol B for inhibiting breast cancer stem cells (Breast CSC).

FIG. 76 shows the cytotoxicity curve of 4-acetyl-antroquinonol B for inhibiting lung cancer stem cells (Lung CSC).

FIG. 77 shows the cytotoxicity curve of 4-acetyl-antroquinonol B for inhibiting colorectal cancer stem cells (CRC CSC).

### SUMMARY OF THE INVENTION

The present invention provides a method for treating the cancer caused by cancer stem cells, which comprises administering to the subject an effective amount of Antrodia camphorata extracts. The present invention also provides a method for treating the cancer, which comprises administering to the subject an effective amount of 4-acetyl-antroquinonol B.

### DETAIL DESCRIPTION OF THE INVENTION

To assess the potential for inhibiting cancer stem cells for lung cancer, brain tumor, head and neck cancer, colorectal cancer and breast cancer, a total of five kinds of A. camphorata extracts were prepared as follows: A. camphorata concentrate (D1), ethyl acetate extracts of A. camphorata concentrate (D2), lyophilized powder of A. camphorata concentrate (D3), ethyl acetate extracts of lyophilized powder of A. camphorata concentrate (D4) and ethyl acetate extracts of A. camphorata mycelium (D5).

The purpose of the present invention is to screen for the A. camphorata extracts with anti-cancer activity. MTT (3-(4,5-dimethylthiazol-2-yl)-2,5-diphenyltetrazolium bromide) assay was used to complete the cytotoxicity test. Human lung cancer stem cells (lung CSC), human Adult fibroblast-1 (AF-1), glioblastoma multiforme cancer stem cells (GBM CSC), human Adult fibroblast-2 (AF-2), head and neck squamous cell carcinoma cancer stem cells (HNSCC CSC), colorectal cancer stem cells (CRC CSC), as well as breast cancer stem cells (Breast CSC), hepatoma cancer stem cells (hepatoma CSC), leukemia cancer stem cells (leukemia CSC) and gastric cancer stem cells (Gastric CSC) are used in cell experiments of the present invention.

The present invention provides a method for treating cancer caused by cancer stem cells in a subject in need thereof, which comprises administering to the subject an effective amount of Antrodia camphorata extracts. The Antrodia camphorata extracts are selected from the group consisting of ethyl acetate extracts of lyophilized powder of Antrodia camphorata concentrate and ethyl acetate extracts of Antrodia camphorata mycelium. The cancer stem cells are selected from liver cancer stem cells, lung cancer stem cells, brain tumor stem cells, head and neck cancer stem cells, colorectal cancer stem cells, breast cancer stem cells, leukemia cancer stem cells or gastric cancer stem cells. In a more preferred embodiment, the liver cancer stem cells are hepatoma cancer stem cells; the brain tumor stem cells are glioblastoma multiforme cancer stem cells; the head and neck cancer stem cells are head and neck squamous cell carcinoma cancer stem cells.

The effective amount of the Antrodia camphorata extracts ranges from 10 μg/ml to 500 μg/ml. In a more preferred embodiment, the effective amount of the Antrodia camphorata extracts ranges from 20 μg/ml to 400 μg/ml. In a most preferred embodiment, the effective amount of the Antrodia camphorata extracts ranges from 40 μg/ml to 300 μg/ml.

The method further comprises co-administration of a chemotherapy drug to increase inhibitory effect of the cancer stem cells. The chemotherapy drug is selected from Cisplatin or Taxol.

The method further comprises co-administration of ionizing radiation to increase inhibitory effect of the cancer stem cells.

The present invention also provides a method for treating cancer caused by cancer stem cells in a subject in need thereof, which comprises administering to the subject an effective amount of 4-acetyl-antroquinonol B. The cancer stem cells are selected from lung adenocarcinoma CD133 positive cancer stem cells, lung cancer stem cells, brain tumor stem cells, breast cancer stem cells, oral cancer stem cells or colorectal cancer stem cells. In a more preferred embodiment, the brain tumor stem cells are glioblastoma multiforme cancer stem cells.

The effective amount of the 4-acetyl-antroquinonol B ranges from 0.1 μg/ml to 100 μg/ml. In a more preferred embodiment, the effective amount of the 4-acetyl-antroquinonol B ranges from 1 μg/ml to 80 μg/ml. In a most preferred embodiment, the effective amount of the 4-acetyl-antroquinonol B ranges from 5 μg/ml to 60 μg/ml.

### EXAMPLES

### Preparation of A. Camphorata Extracts

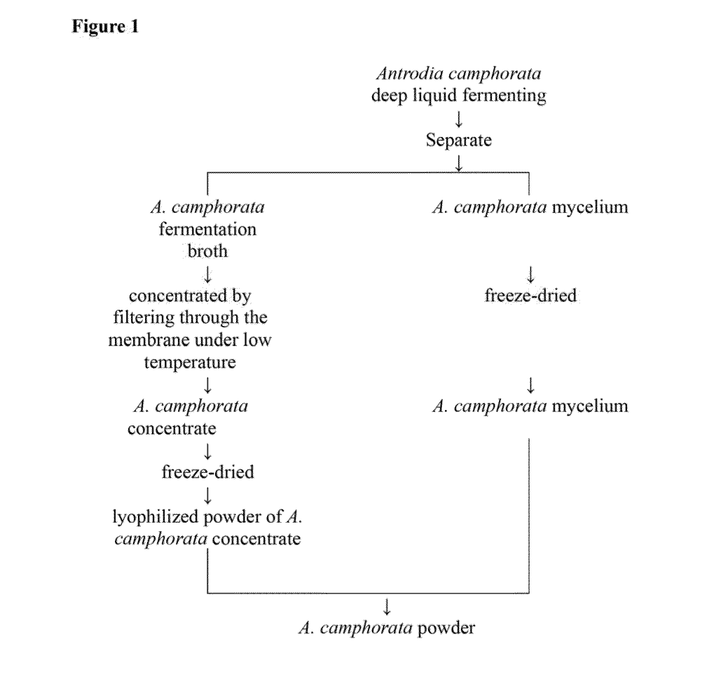

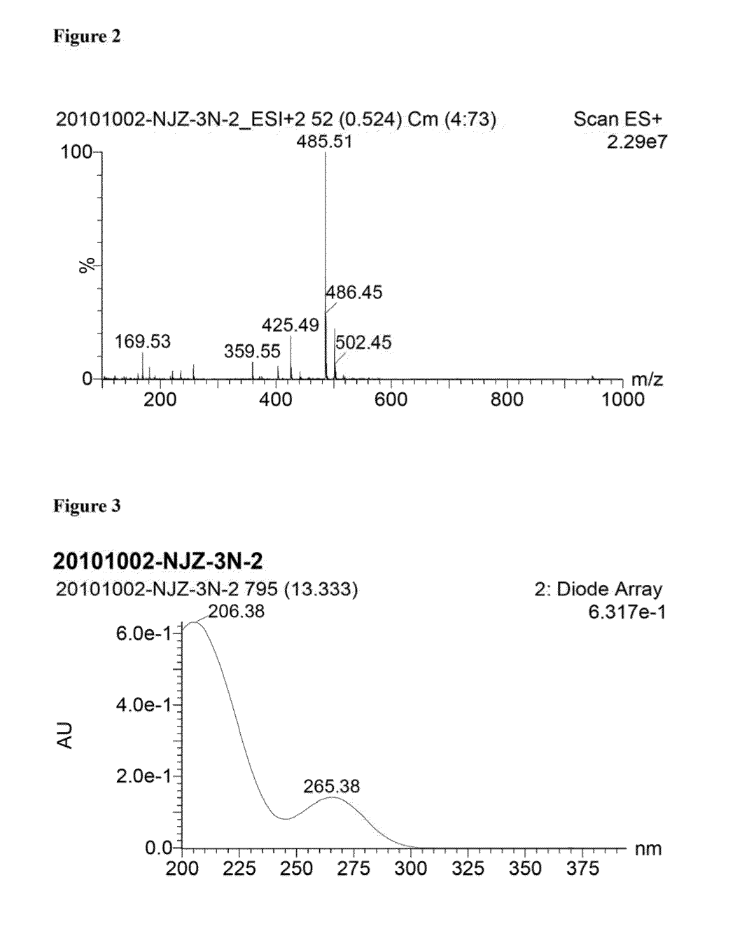

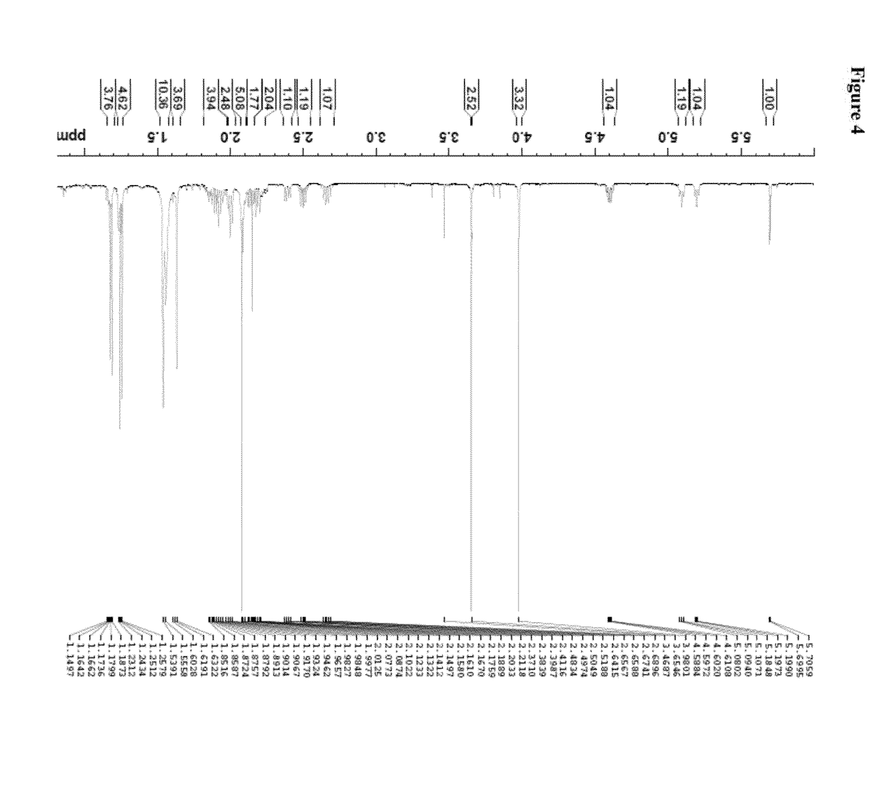

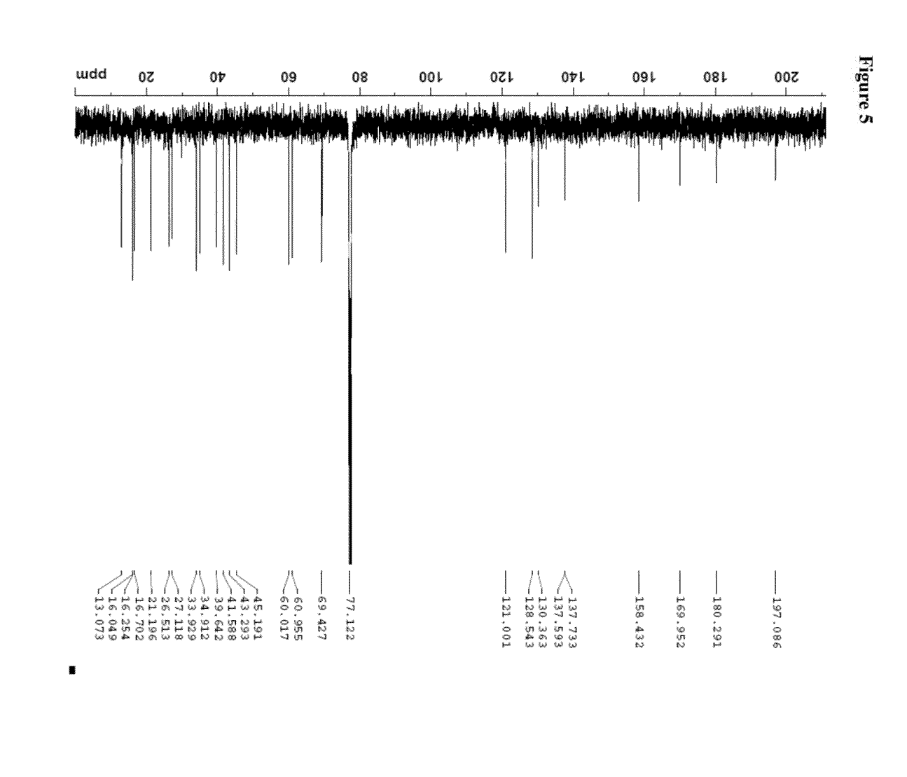

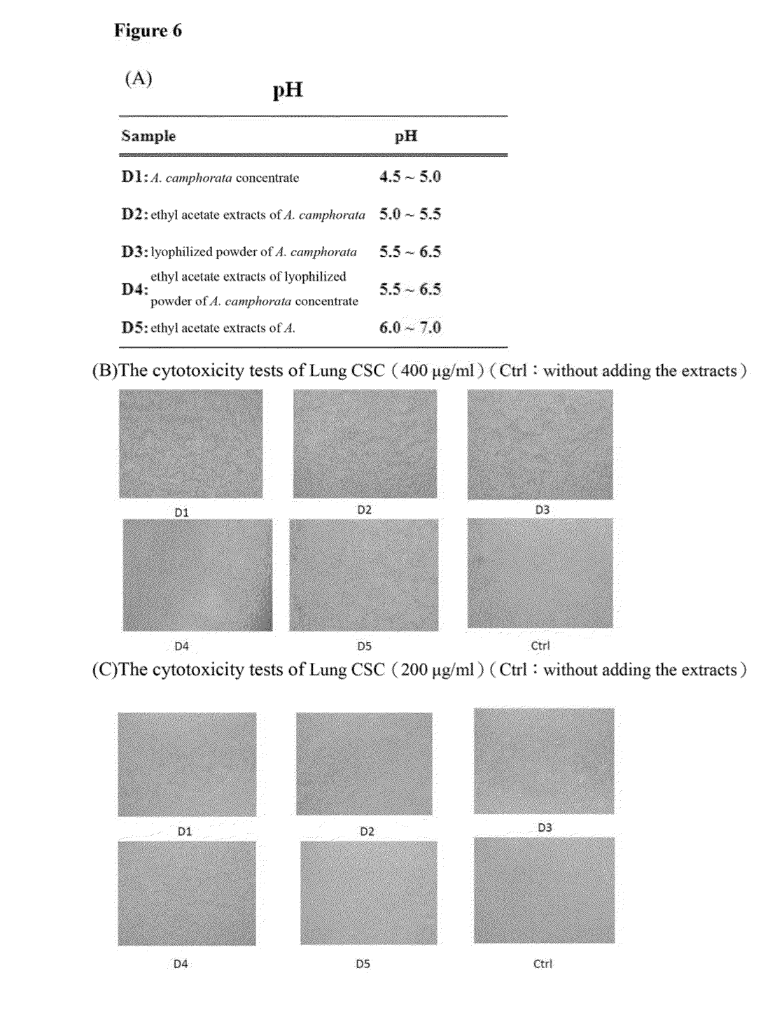

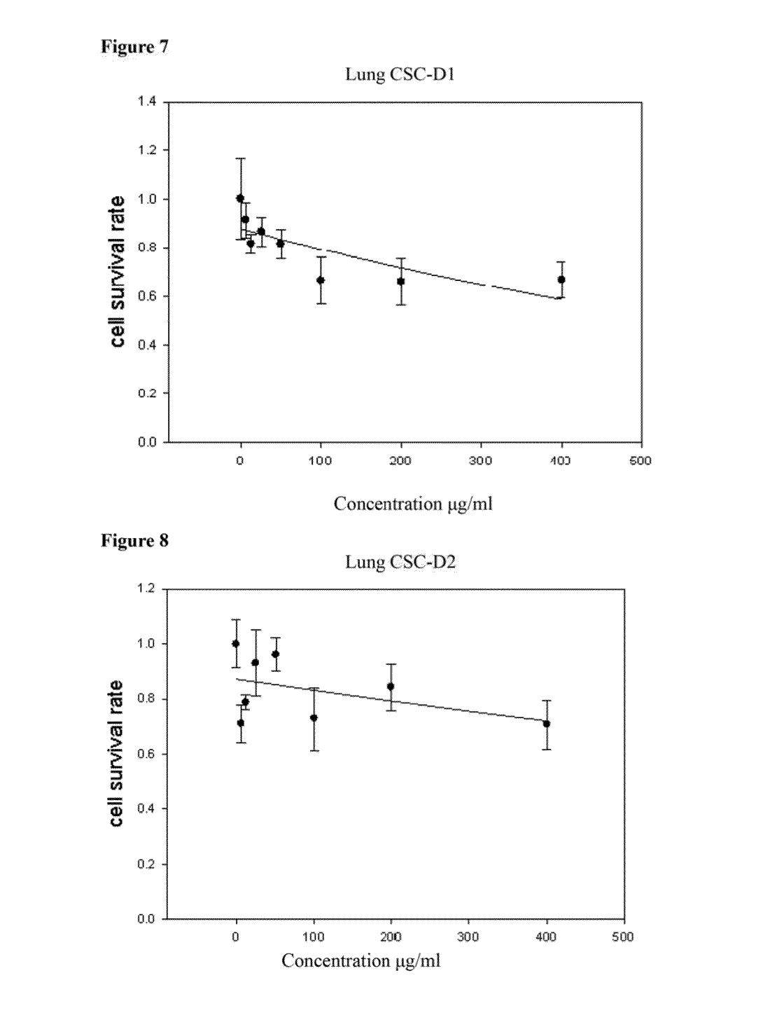

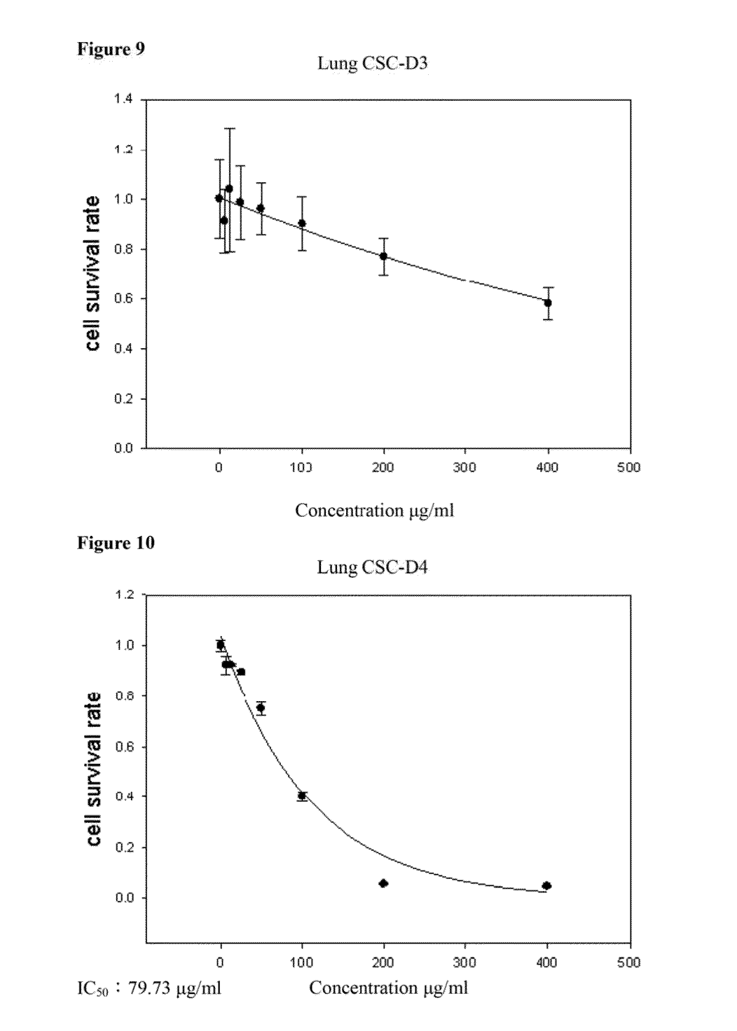

The A. camphorata was incubated to produce A. camphorata fermented concentrate. The production process of A. camphorata was shown in FIG. 1 and a total of five A. camphorata extracts were prepared as follows:
1. A. camphorata concentrate (D1);
2. ethyl acetate extracts of A. camphorata concentrate (D2);
3. lyophilized powder of A. camphorata concentrate (D3);
4. ethyl acetate extracts of lyophilized powder of A. camphorata concentrate (D4);
5. ethyl acetate extracts of A. camphorata mycelium (D5)

The A. camphorata was incubated to generate A. camphorata fermentation broth and then was concentrated by filtering through the membrane under low temperature to generate A. camphorata concentrate (D1) and the A. camphorata concentrate were further freeze-dried to generate the lyophilized powder of A. camphorata concentrate (D3).

Preparation of ethyl acetate extracts of A. camphorata concentrate (D2): 100 ml of A. camphorata concentrate was added into 100 ml of ethyl acetate to partition (3 times). The ethyl acetate layer was collected, concentrated and dried to generate the ethyl acetate extracts.

Preparation of ethyl acetate extracts of lyophilized powder of A. camphorata concentrate (D4): 10 g of lyophilized powder of A. camphorata concentrate was added into 100 ml of 95% ethanol for reflux extraction for 3 hours. After sample was concentrated and dried, 100 ml of water and 100 ml of ethyl acetate were added to partition (3 times). The ethyl acetate layer was collected, concentrated and dried to generate the ethyl acetate extracts of lyophilized powder of A. camphorata concentrate.

Preparation of the ethyl acetate extracts of A. camphorata mycelium (D5): 10 g of A. camphorata mycelium was added into 100 ml of 95% ethanol for reflux extraction for 3 hours. After sample was concentrated and dried, 100 ml of water and 100 ml of ethyl acetate were added to partition (3 times). The ethyl acetate layer was collected, concentrated and dried to generate the ethyl acetate extracts of A. camphorata mycelium.

Isolation of the 4-acetyl-antroquinonol B of the Ethyl Acetate Extracts of A. Camphorata Mycelium (D5)

3 kg of A. camphorata mycelium was added to 10 L of 95% of ethanol and heated for reflux extraction for 4 times. The extract was filtered and concentrated, then dried under reduced pressure to generate 384 g of ethanol extracts. The ethanol extracts was suspended with water and partitioned with equal amount of ethyl acetate. The ethyl acetate layer was concentrated under reduced pressure to obtain 157.57 g of ethyl acetate layer partition and 159.51 g of water layer partition.

The 157.57 g of ethyl acetate layer partition was chromatographed on silica gel column (10 cm i.d×30 cm). Following the order of n-hexane→n-hexane-ethyl acetate (10:1→10:2→10:3→10:4→10:5→1:1→1:2, v/v)→ethyl acetate→methanol, 10 L of each proportion were used to elute and each 1 L was collected as a partition. The eluted partition 56-63(3.015 g) of n-hexane-ethyl acetate (10:4) was chromatographed using reversed phase preparative column (Tosoh ODS-80Ts, 21.5 mm×300 mm, 10 μm). H₂O—CH₃CN (20:80) was used as the mobile phase at a flow rate of 10 ml/min for chromatography, and the detecting wavelength was 265 nm, the column temperature was fixed at 40° C. 4-acetyl-antroquinonol B (131 mg) was obtained.

Identification of the Structure of 4-acetyl-antroquinonol B

4-acetyl-antroquinonol B, ESI(+)MS (m/z): 485 [M+Na]⁺, 502 [M+K]⁺. UV λₘₐₓ (nm): 206, 265. ¹H-NMR (500 MHz, CDCl₃) δ (ppm): 5.70 (1H, d, J=3.2 Hz, H-4), 5.20 (1H, t, J=6.4 Hz, H-12), 5.09 (1H, t, J=6.8 Hz, H-8), 4.60 (1H, m, H-15), 3.98 (3H, s, H-24), 3.65 (3H, s, H-23), 2.67 (1H, m, H-17), 2.50 (1H, dq, J=6.9, 10.8 Hz, H-6), 2.39 (1H, dd, J=6.5, 13.9 Hz, H-14), 2.23 (1H, m, H-11), 2.19 (1H, m, H-14), 2.14 (1H, m, H-16), 2.09 (2H, m, H-7), 2.08 (3H, OAc), 2.00 (2H, m, H-10), 1.94 (1H, m, H-11), 1.90 (1H, m, H-16), 1.86 (1H, m, H-5), 1.63 (3H, s, H-21), 1.54 (3H, s, H-20), 1.25 (3H, d, J=7.3 Hz, H-19), 1.18 (3H, d, J=6.9 Hz, H-22). ¹³C-NMR(125 MHz, CDCl₃) δ (ppm): 197.1 (C-1), 180.3 (C-18), 170.0 (CH₃CO), 158.4 (C-3), 137.7 (C-9), 137.6 (C-2), 130.4 (C-13), 128.5 (C-12), 121.0 (C-8), 77.1 (C-15), 69.4 (C-4), 61.0 (C-23), 60.0 (C-24), 45.2 (C-14), 43.3 (C-5), 41.6 (C-6), 39.6 (C-10), 34.9 (C-16), 33.9 (C-17), 27.1 (C-11), 26.5 (C-7), 21.2 (CH₃CO), 16.7 (C-21), 16.3 (C-20), 16.1 (C-19), 13.1 (C-22).

### Drug Toxicity Tests

In the present embodiment, a specific number of cells were cultured in 25T cell culture medium. After 6 hours, the cells attached to the bottom of the medium. Drugs (A. camphorata extracts) of 0, 50, 100, 200, and 400 nM were added when the cells remained in undivided state. The medium containing the drugs were removed at 0, 6, 12 and 24 hours after incubation. Cells were washed with PBS once and replenished with the drug-free fresh medium, then cultured for 10 to 14 days. The cultured cells were fixed with methanol and stained with Giemsa, and then the number of cell colonies were counted (each colony must contain more than 50 cells). The surviving fractions (SF) were calculated as:

$${SF}_{xnM , thr} = \frac{{PE}_{xnM , thr}}{{PE}_{onM , thr}} ( SF \text{:} surviving fraction , PE \text{:} plating efficiency )$$

Combining with Radiation Treatment

A specific number of cells were cultured in 25T cell culture medium. After the cells attached to the bottom of the medium, the medium was replaced and 200 nM of the fresh cell culture medium was added. Cells were cultured for 24 hours and then irradiated with radiation. The cell culture medium was replaced immediately after irradiation. The cells were cultured for further 10 to 14 days and then stained with Giemsa. The number of cell colonies (each colony must contain more than 50 cells) were counted. The surviving fractions (SF) were calculated as:

$${SF}_{xnM , DGy} = \frac{{PE}_{xnM , DGy}}{{PE}_{onM , DGy}} ( SF \text{:} surviving fraction , PE \text{:} Plating efficiency )$$

Different cancer stem cells were used to test for cytotoxic concentration of co-administration of radiation therapy with five A. camphorata extracts based on the half maximal inhibitory concentration (IC₅₀) of D1˜D5.

### Cytotoxic Activity Test (MTT Assay)

The principle of cytotoxic activity test (MTT assay) is that succinate dehydrogenase in the mitochondria of a living cell can reduce MTT (3-(4,5-dimethylthiazol-2-yl)-2,5-diphenyltetrazolium bromide), and blue-violet formazan was formed under reaction with cytochrome C. Generally, the amount of formazan generated is proportional to the activity of the mitochondria and the number of living cells. After adding dimethyl sulfoxide (DMSO) to dissolve formazan, the number of living cells can be estimated by the optical density (OD) value.

### Half Maximal Inhibitory Concentration (IC₅₀)

The definition of half maximal inhibitory concentration (IC₅₀) is the concentration for survival rate of 50% under the reaction with drugs or compounds.

### Chemotherapy Drug Standards

Cisplatin: cis-diammineplatinum(II) dichloride (Sigma-Aldrich, USA)

Taxol: Paclitaxel (Sigma-Aldrich, USA)

Combining with Chemotherapy Drugs

According to the pretested half maximal inhibitory concentration (IC₅₀) of D1˜D5, different cancer stem cells were used to test the cytotoxic concentration for (1) the co-administration of A. camphorata extracts with the chemotherapy drug, Cisplatin; (2) the co-administration of A. camphorata extracts with the chemotherapy drug, Taxol.

### The Inhibitory Ability of A. Camphorata Extracts for Tumor Cells

To assess the ability of A. camphorata extracts against lung cancer, brain cancer, head and neck cancer, colorectal cancer, and breast cancer for inhibiting the growth of cancer stem cells, five A. camphorata extracts were tested as follows: A. camphorata concentrate (D1), ethyl acetate extracts of A. camphorata concentrate (D2), lyophilized powder of A. camphorata concentrate (D3), ethyl acetate extracts of lyophilized powder of A. camphorata concentrate (D4), ethyl acetate extracts of A. camphorata mycelium (D5). The aim of this embodiment was to screen for the A. camphorata extracts with anti-cancer effect. MTT (3-(4,5-dimethylthiazol-2-yl)-2,5-diphenyltetrazolium bromide) assay was used to conduct the cytotoxicity test. Since the dead cells do not have succinate dehydrogenase, there is no reaction after adding MTT. Two human fibroblast cells, AF-1 (Adult fibroblast-1) and AF-2 (Adult fibroblast-2), human lung cancer stem cell (Lung CSC), Glioblastomas multiform cancer stem cells (GBM CSC), Head and neck squamous cell carcinoma cancer stem cells (HNSCC CSC), colorectal cancer stem cells (CRC CSC), breast cancer stem cells (Breast CSC) were used as cell experimental models.

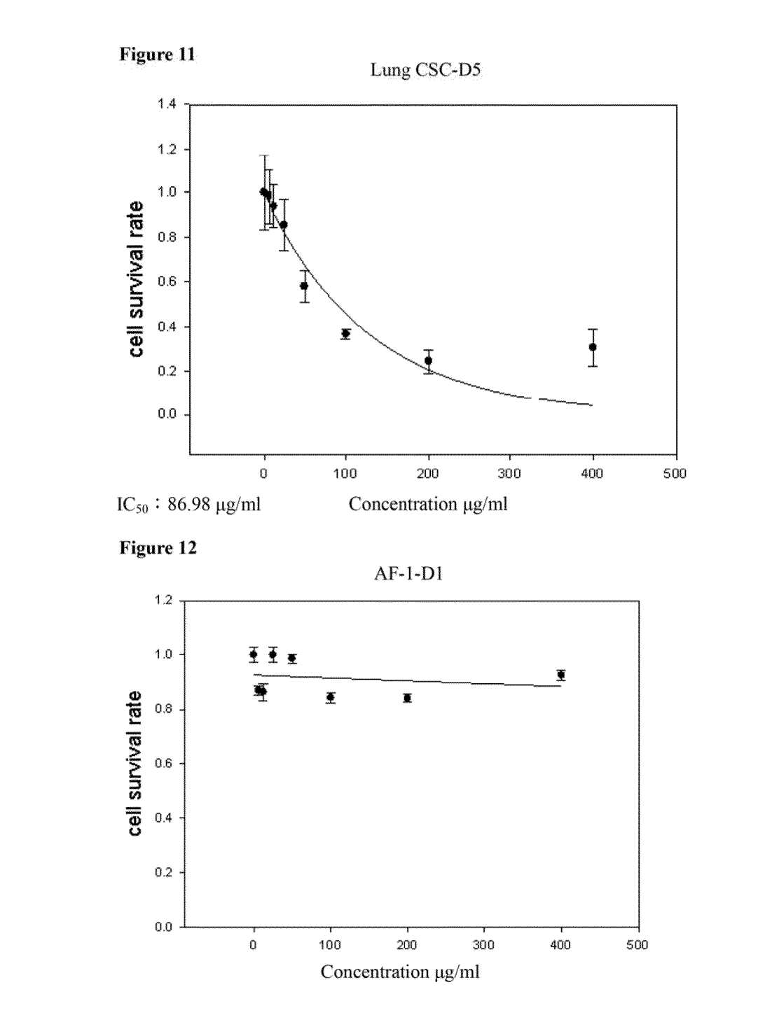

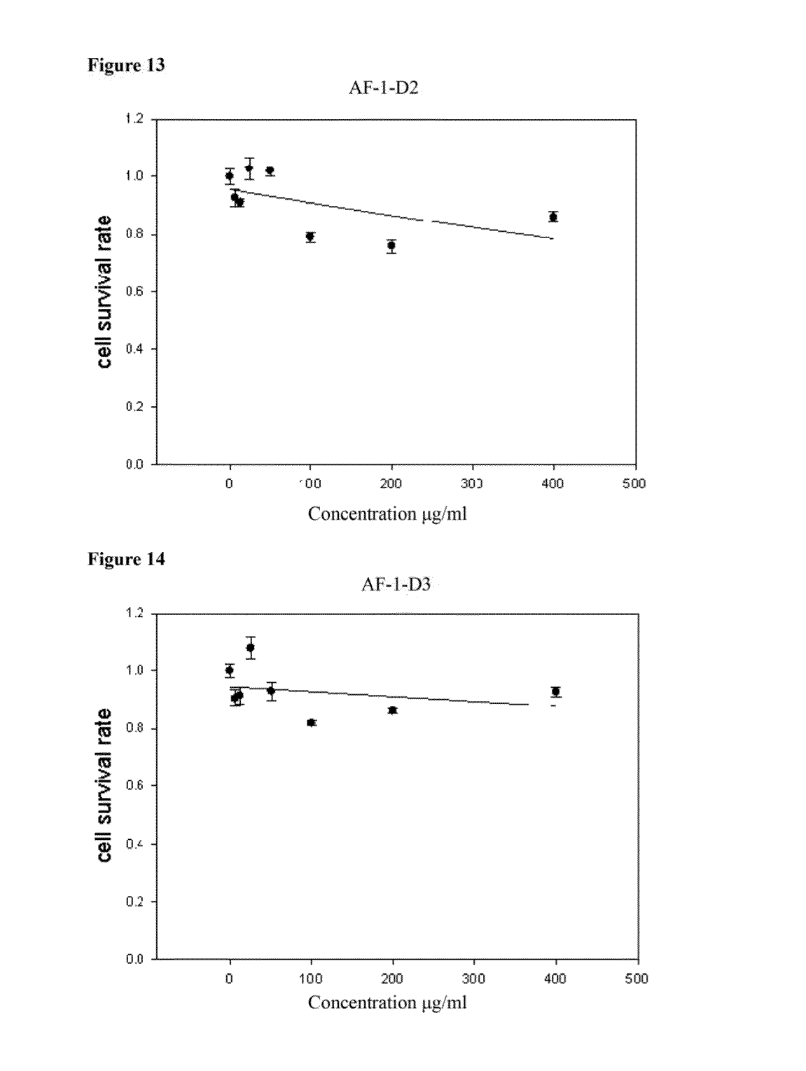

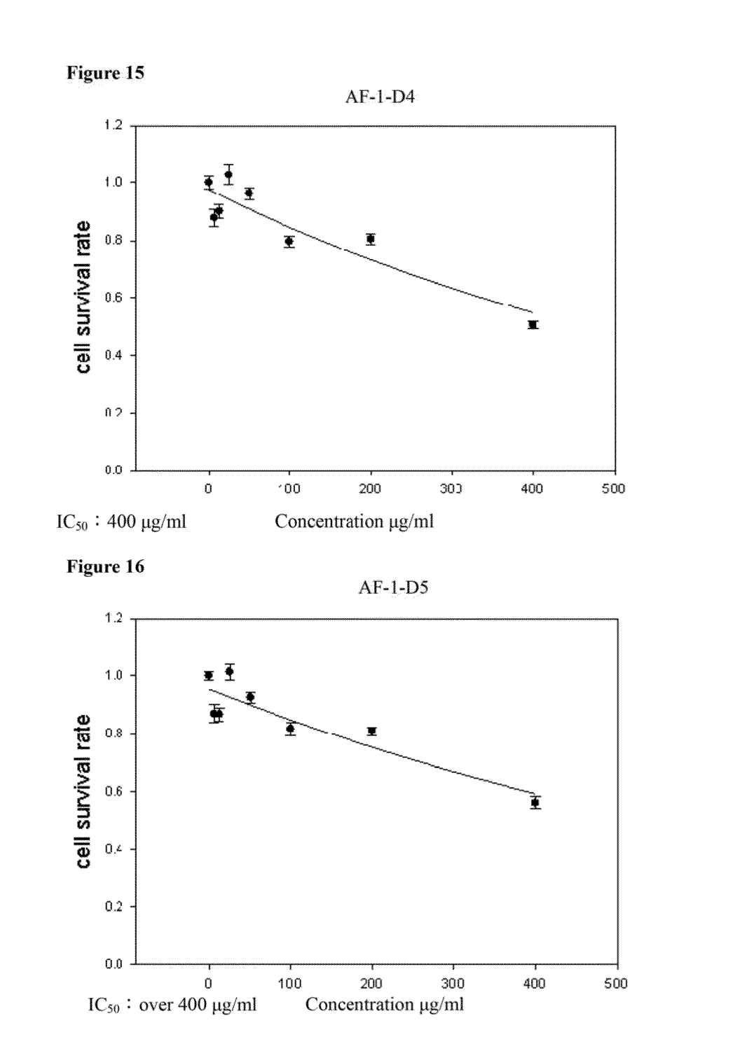

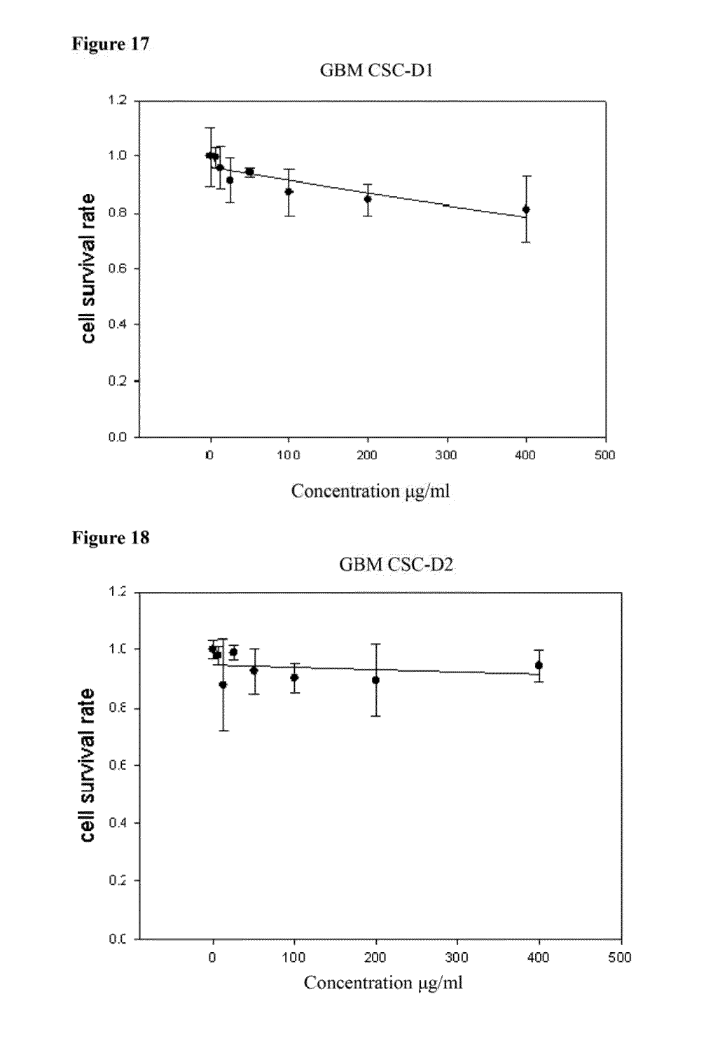

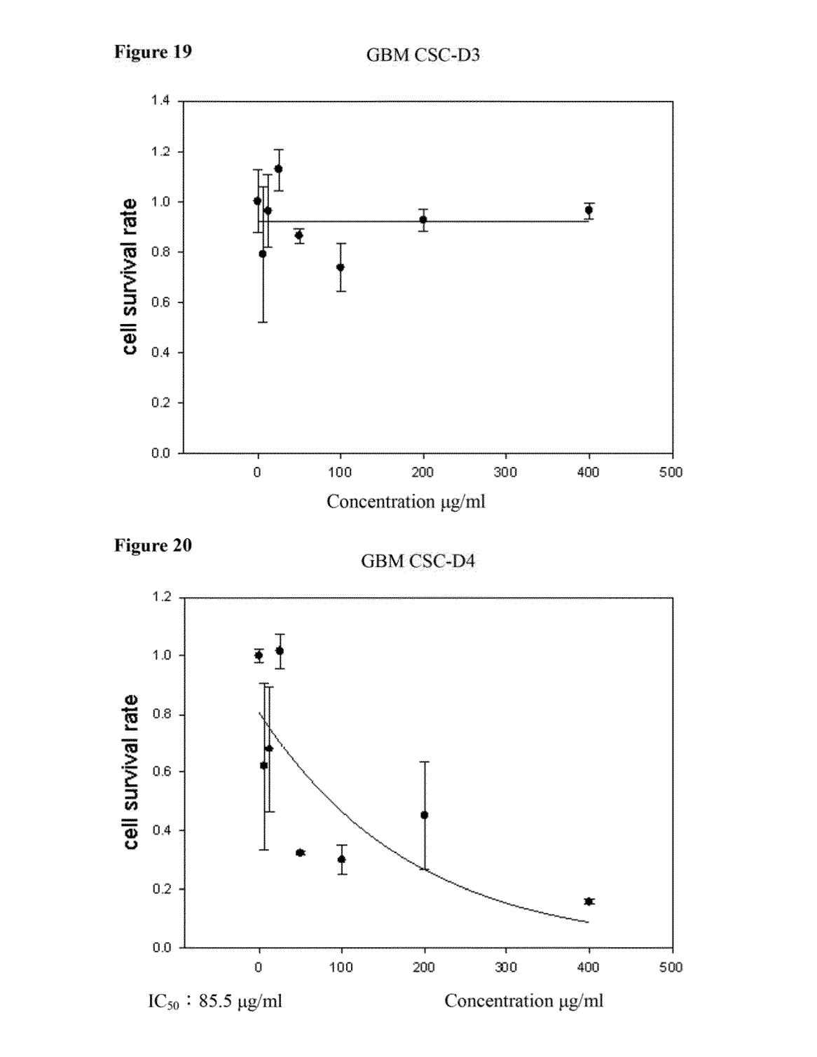

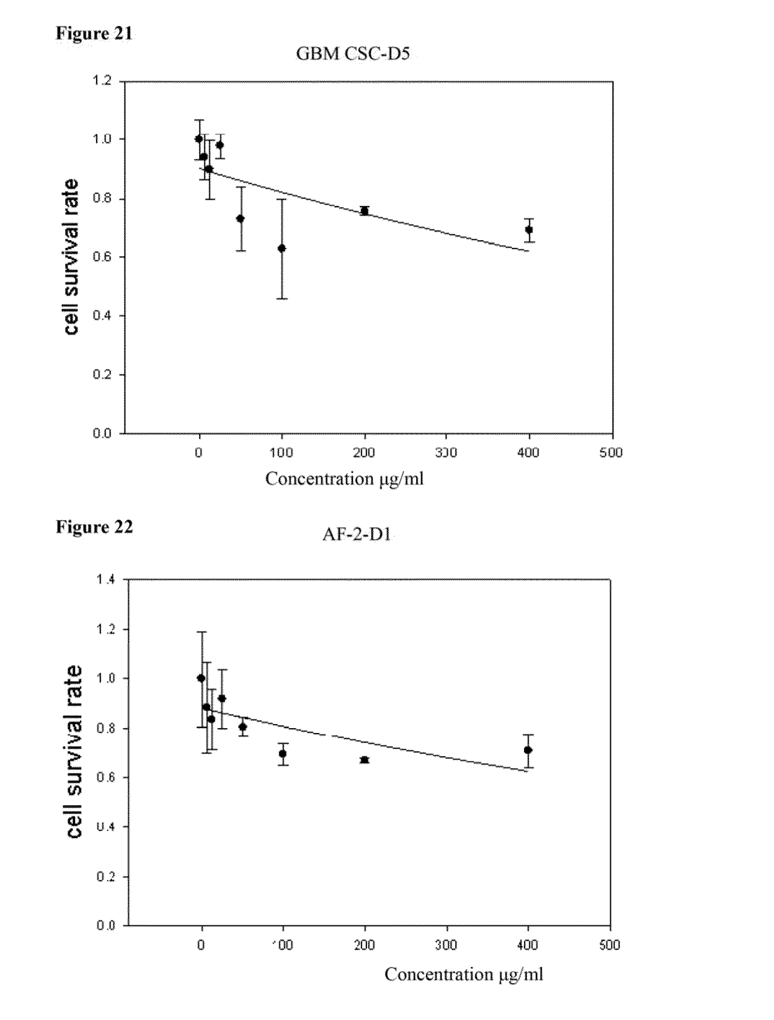

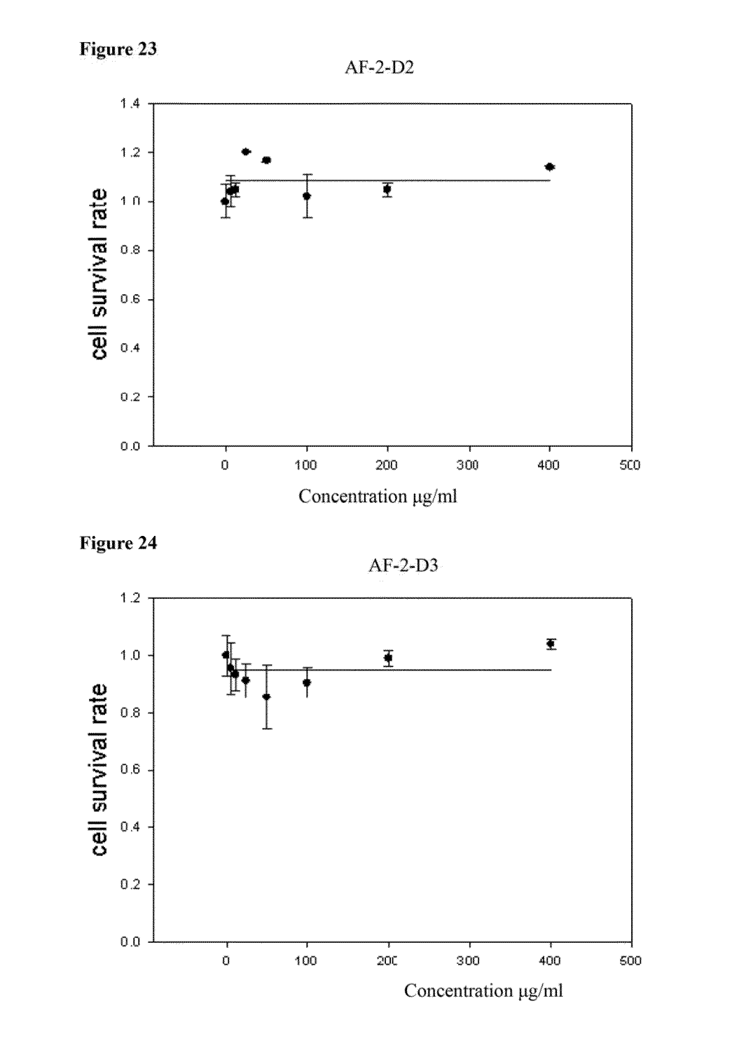

In the initial screening results of A. camphorata extracts, ethyl acetate extracts of lyophilized powder of A. camphorata concentrate (D4) and ethyl acetate extracts of A. camphorata mycelium (D5) had the best growth inhibitory effects for human lung cancer stem cell (Lung CSC), as shown in FIGS. 10, 11; ethyl acetate extracts of lyophilized powder of A. camphorata concentrate (D4) and ethyl acetate extracts of A. camphorata mycelium had the best growth inhibitory effects for human fibroblast cells AF-1 (Adult fibroblast-1), as shown in FIGS. 15, 16. In drug screening results, half maximal inhibitory concentration (IC₅₀) was shown in Table 1 that ethyl acetate extracts of lyophilized powder of A. camphorata concentrate (D4) and ethyl acetate extracts of A. camphorata mycelium (D5) had better growth inhibitory effects.

TABLE 1
The half maximal inhibitory concentration (IC₅₀) of A. camphorata
extracts for human fibroblast cells AF-1(Adult fibroblast-1),
AF-2 (Adult fibroblast-2), lung cancer stem cells (Lung CSC),
Glioblastomas multiform cancer stem cells (GBM CSC), Head and
neck squamous cell carcinoma cancer stem cells (HNSCC CSC),
colorectal cancer stem cells (CRC CSC), breast cancer stem cells
(Breast CSC), Hepatoma cancer stem cells (Hepatoma CSC),
Leukemia cancer stem cells (Leukemia CSC) and Gastric
stem cells (Gastric CSC).
IC₅₀

| | D1 | D2 | D3 | D4 | D5 | |
|---|---|---|---|---|---|---|
| Adult fibroblast (AF-1) | x | x | x | 400 μg/ml | Over 400 | μg/ml |
| Adult fibroblast (AF-2) | x | x | x | 164.6 μg/ml | 224.3 | μg/ml |
| Lung cancer stem cells | x | x | x | 79.7 μg/ml | 86.9 | μg/ml |
| (Lung CSC) | | | | | | |
| Hepatoma cancer stem | x | x | x | 167.8 μg/ml | 268.7 | μg/ml |
| cells (Hepatoma CSC) | | | | | | |
| Colorectal cancer stem | x | x | x | 173.4 μg/ml | 191.2 | μg/ml |
| cells (CRC CSC) | | | | | | |
| Breast cancer stem cells | x | x | x | 55.8 μg/ml | 168.6 | μg/ml |
| (Breast CSC) | | | | | | |
| Leukemia cancer stem | x | x | x | 123.7 μg/ml | 258.9 | μg/ml |
| cells (Leukemia CSC) | | | | | | |
| Gastric cancer stem cells | x | x | x | 119.3 μg/ml | 210.5 | μg/ml |
| (Gastric CSC) | | | | | | |
| Glioblastomas multiform | x | x | x | 85.5 μg/ml | x | |
| cancer stem cells | | | | | | |
| (GBM CSC-1) | | | | | | |
| Glioblastomas multiform | x | x | x | 132.6 μg/ml | 287.8 | μg/ml |
| stem cells (GBM | | | | | | |
| CSC-2) | | | | | | |
| Head and neck squamous | x | x | x | 50.9 μg/ml | 97.7 | μg/ml |
| cell carcinoma cancer | | | | | | |
| stem cells (HNSCC | | | | | | |
| CSC) | | | | | | |

“x”: The maximum drug concentration 400 μg/ml still can not effectively inhibit more than 50% of the cells

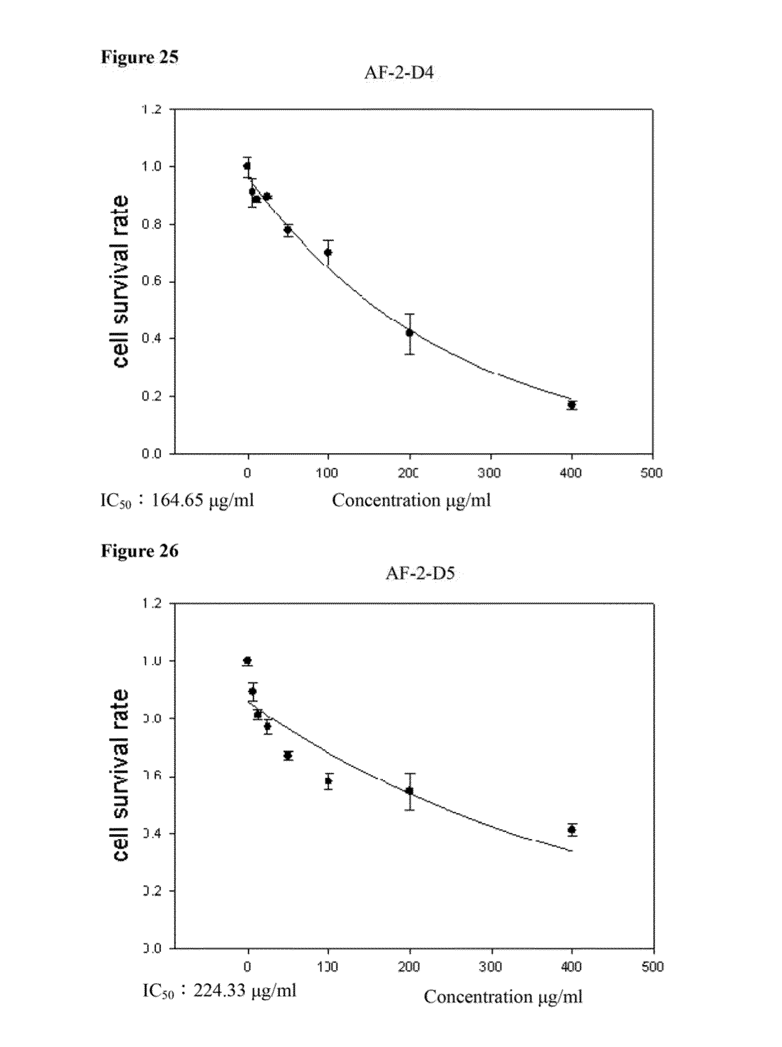

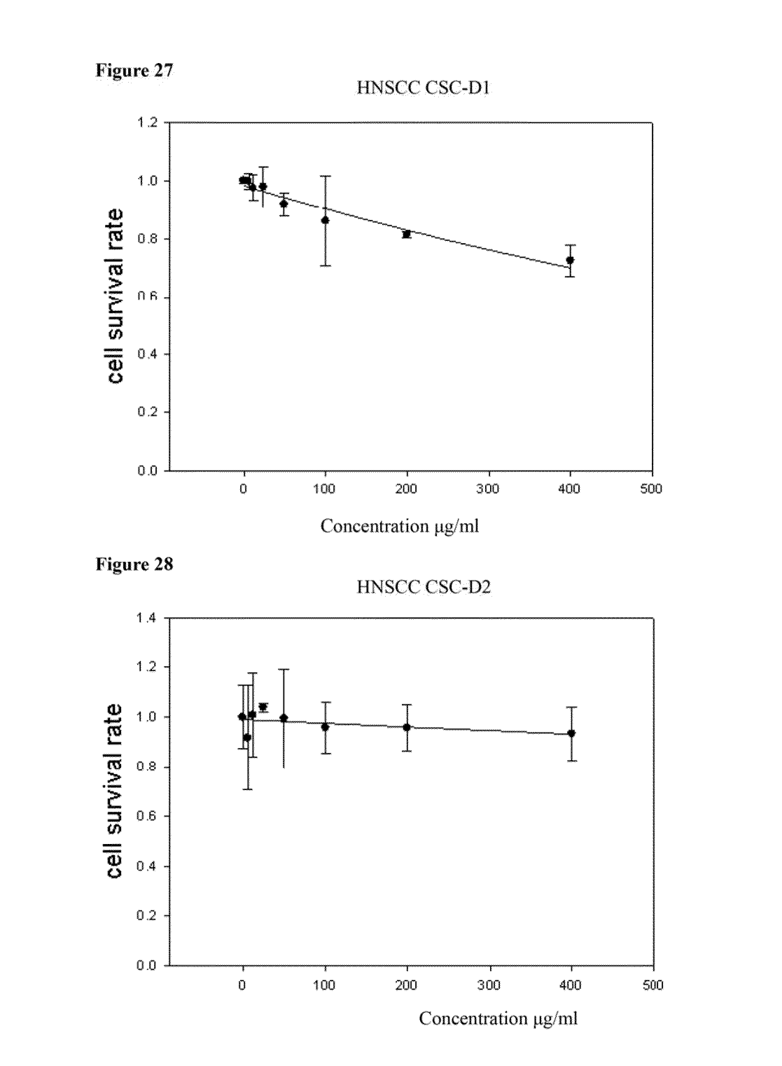

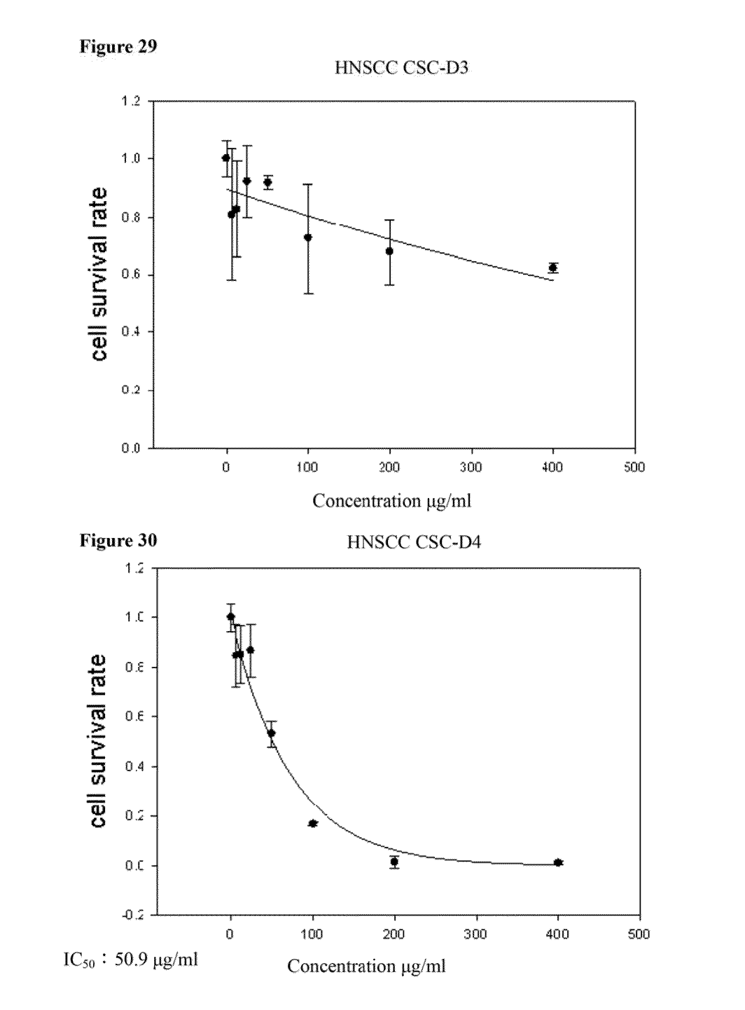

For glioblastoma multiform cancer stem cells, ethyl acetate extracts of lyophilized powder of A. camphorata concentrate (D4) had the best growth inhibitory effects, as shown in FIG. 20; for Adult fibroblast-2 (AF-1), ethyl acetate extracts of lyophilized powder of A. camphorata concentrate (D4) and ethyl acetate extracts of A. camphorata mycelium (D5) had the best growth inhibitory effects, as shown in FIGS. 25, 26.

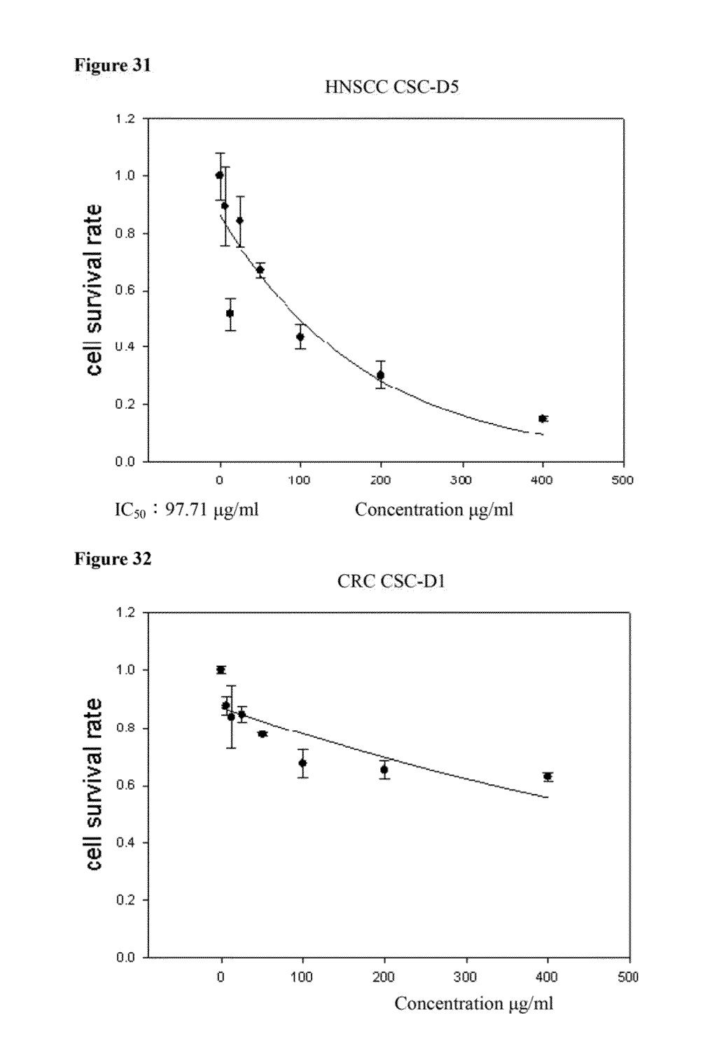

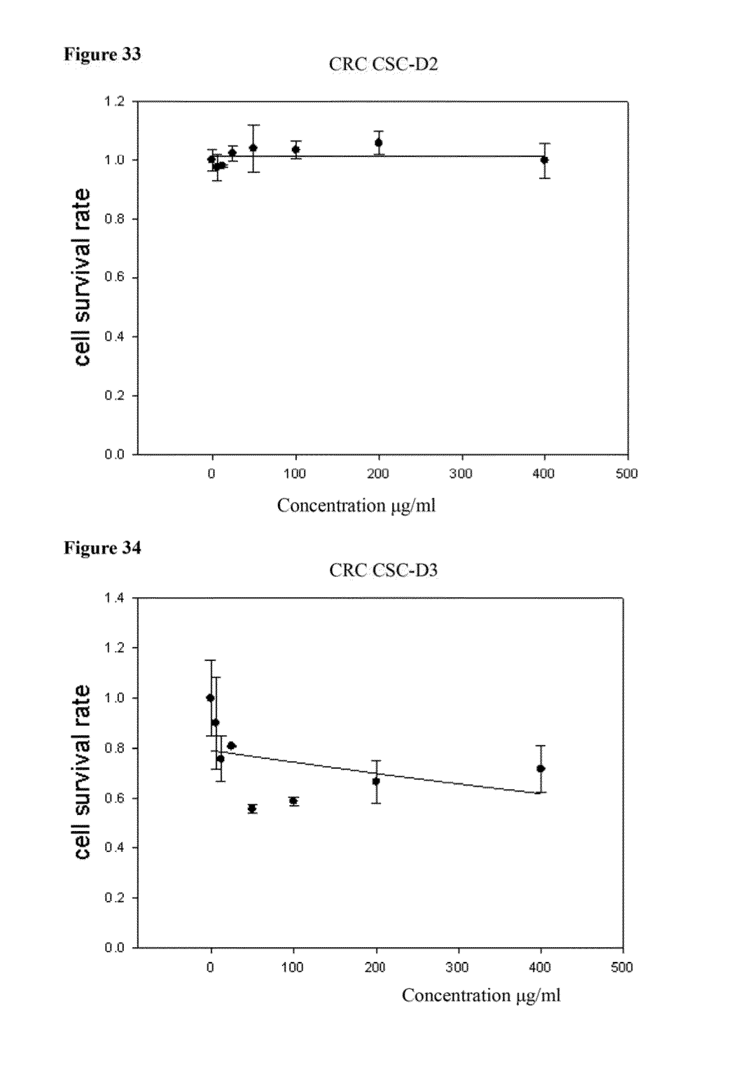

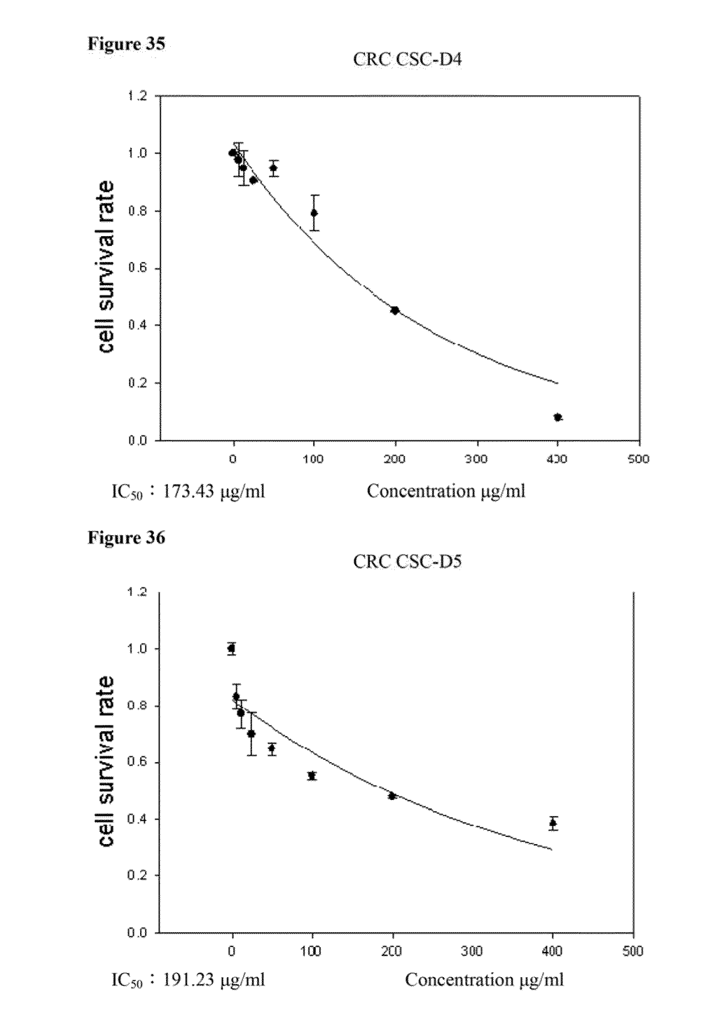

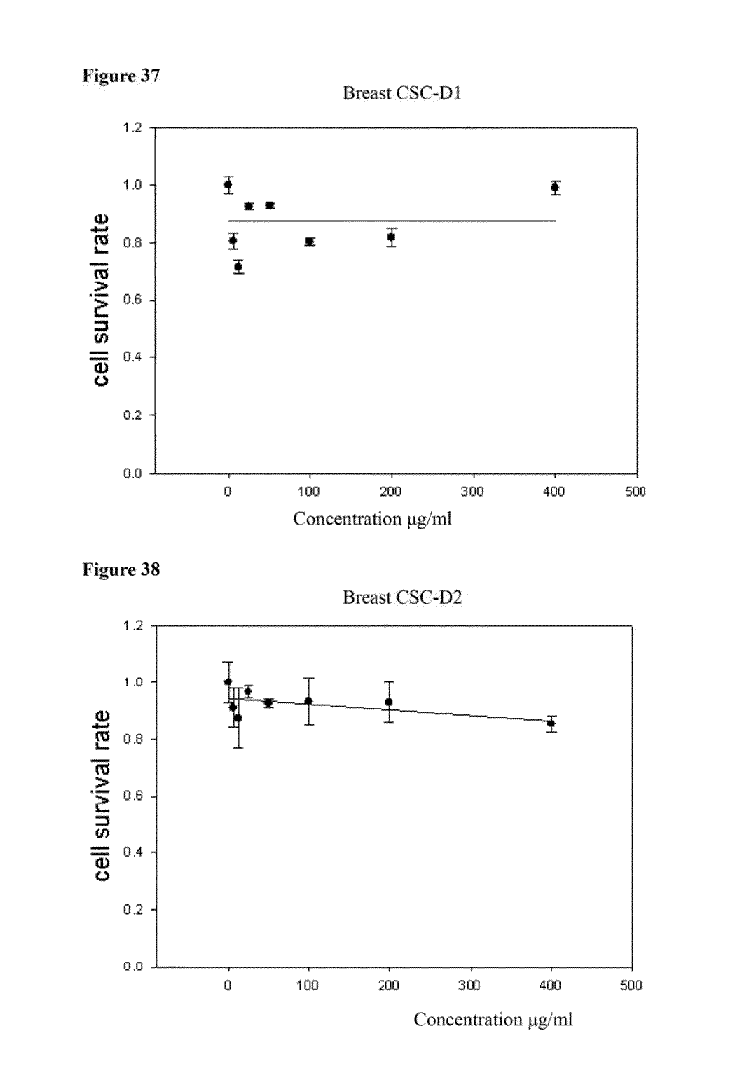

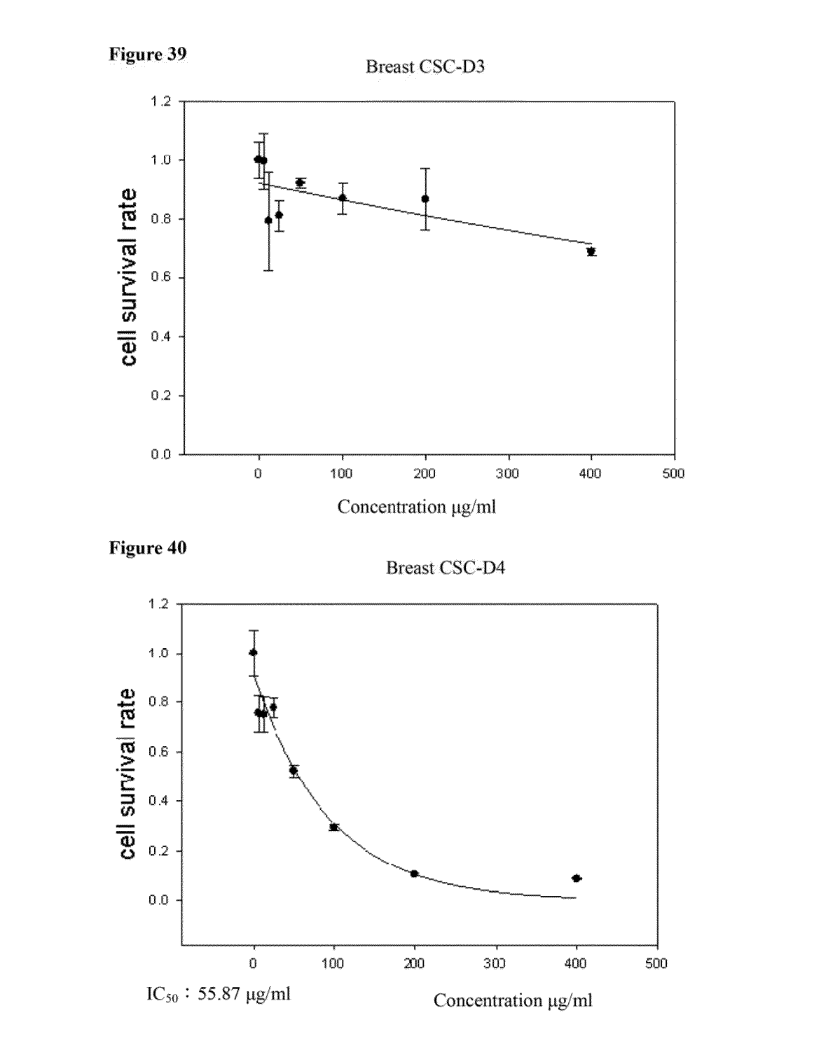

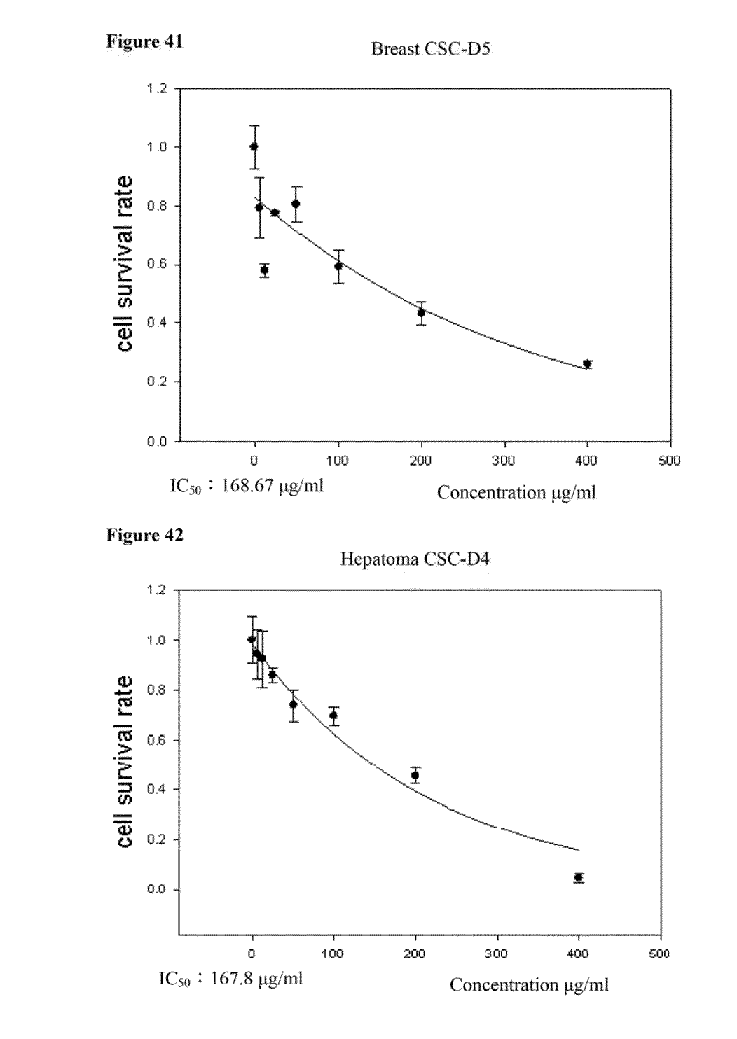

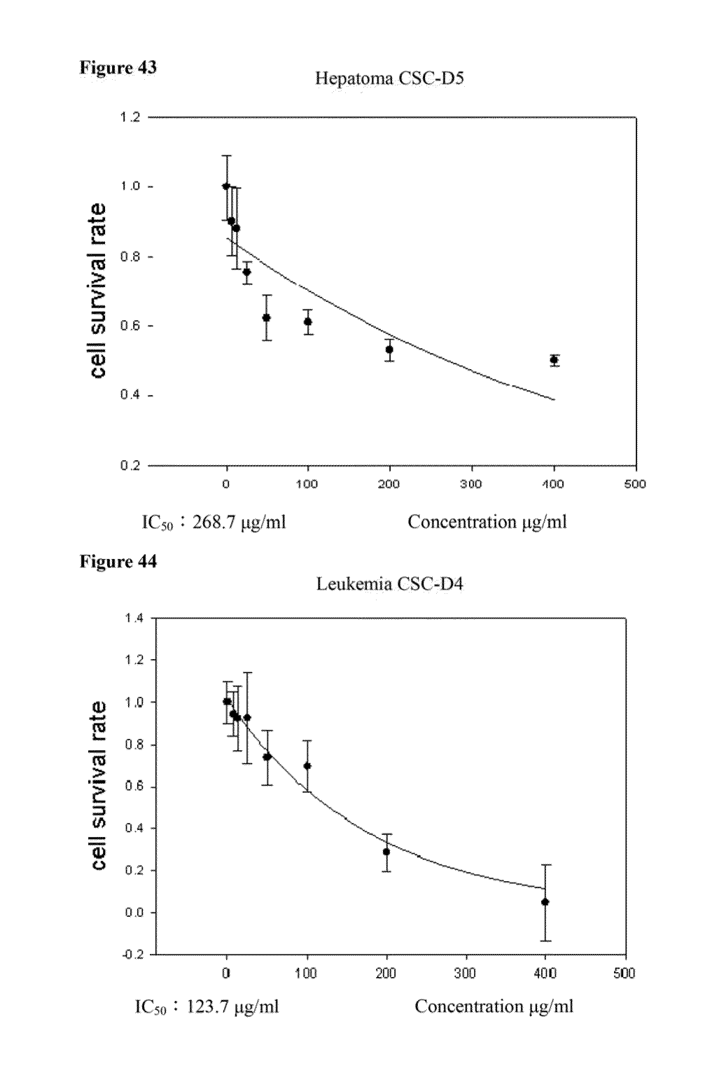

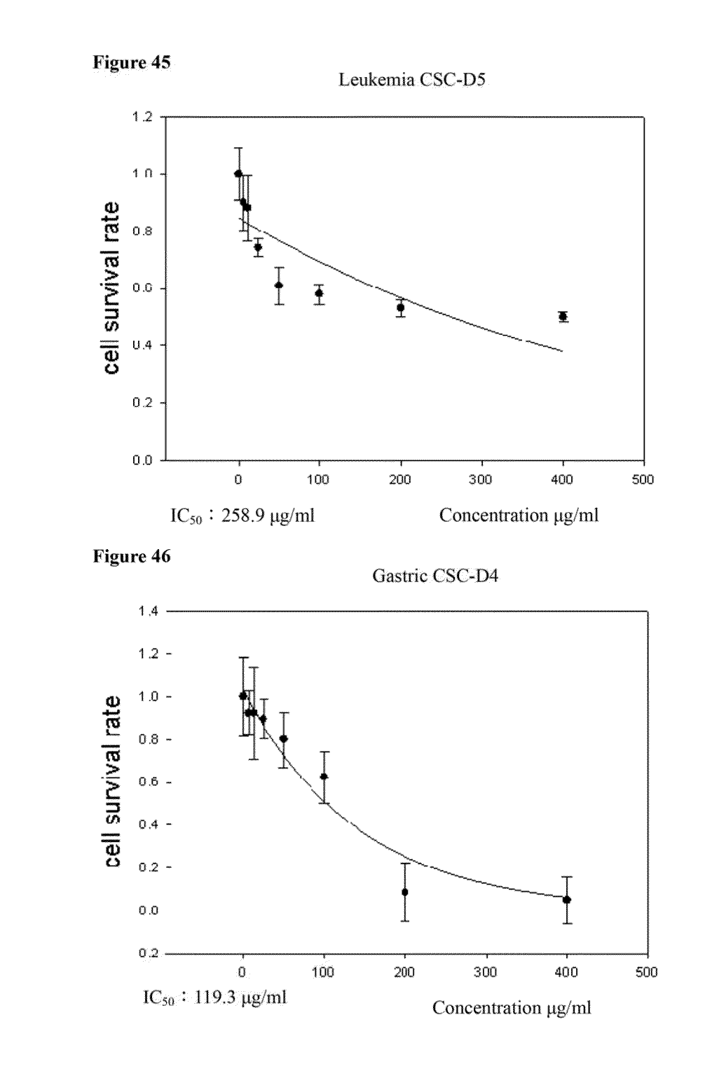

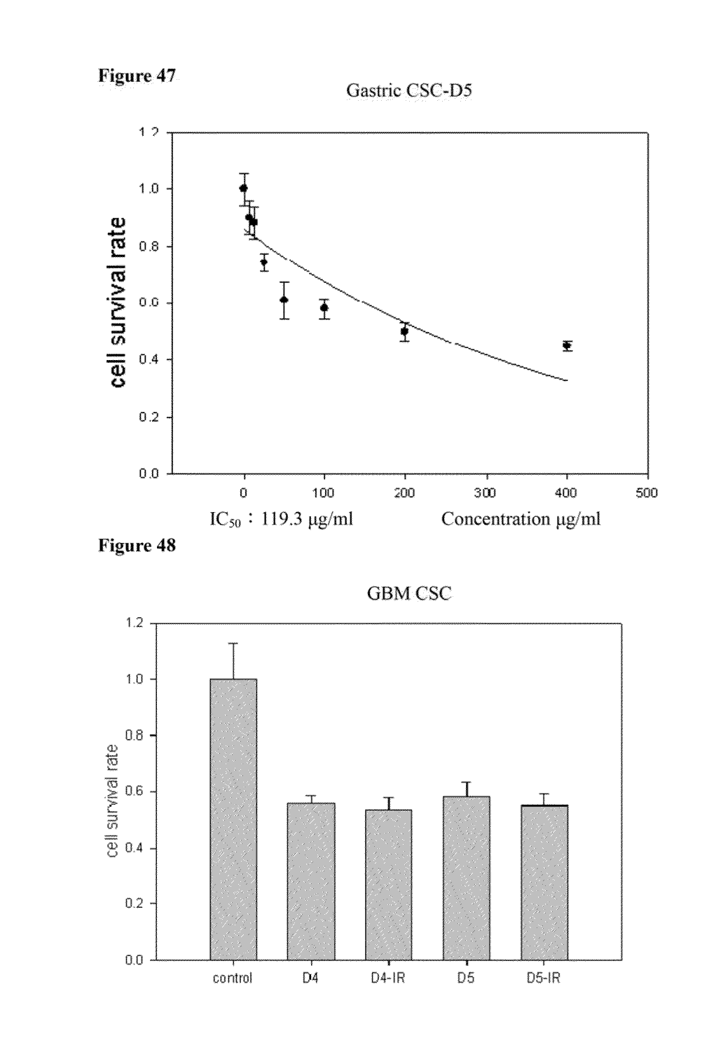

For head and neck squamous cell carcinoma cancer stem cells (HNSCC CSC), ethyl acetate extracts of lyophilized powder of A. camphorata concentrate (D4) and ethyl acetate extracts of A. camphorata mycelium (D5) had the best growth inhibitory effects, as shown in FIGS. 30, 31. For colorectal cancer stem cells (CRC CSC), ethyl acetate extracts of lyophilized powder of A. camphorata concentrate (D4) and ethyl acetate extracts of A. camphorata mycelium (D5) had the best inhibitory growth effects, as shown in FIGS. 35, 36. For Breast cancer stem cells (Breast CSC), ethyl acetate extracts of lyophilized powder of A. camphorata concentrate (D4) and ethyl acetate extracts of A. camphorata mycelium (D5) had the best growth inhibitory effects, as shown in FIGS. 40, 41.

In summary, with respect to the half maximal inhibitory concentration (IC₅₀) of the five A. camphorata extracts (D1 [symbol] D2 [symbol] D3 [symbol] D4 [symbol] D5) for human fibroblast cells AF-1 (Adult fibroblast-1), AF-2 (Adult fibroblast-2), lung cancer stem cells (Lung CSC), Glioblastomas multiform cancer stem cells (GBM CSC), Head and neck squamous cell carcinoma cancer stem cells (HNSCC CSC), colorectal cancer stem cells (CRC CSC), breast cancer stem cells (Breast CSC), ethyl acetate extracts of lyophilized powder of A. camphorata concentrate (D4) and ethyl acetate extracts of A. camphorata mycelium (D5) had the better growth inhibitory effects, as shown in Table 1.

In Table 1, the inhibitory effects of the growth of lung cancer stem cells (Lung CSC), Glioblastomas multiform cancer stem cells (GBM CSC), Head and neck squamous cell carcinoma cancer stem cells (HNSCC CSC), breast cancer stem cells (Breast CSC) were sensitive to ethyl acetate extracts of lyophilized powder of A. camphorata concentrate (D4) and ethyl acetate extracts of A. camphorata mycelium (D5) which had significant growth inhibitory effects on these four kinds of cancer stem cells. However, in comparison to the cancer stem cells, ethyl acetate extracts of lyophilized powder of A. camphorata concentrate (D4) and ethyl acetate extracts of A. camphorata mycelium (D5) had no significant growth inhibitory effects on normal human fibroblast cells AF-1 (Adult fibroblast-1) and AF-2 (Adult fibroblast-2).

According to the results of the present embodiment, ethyl acetate extracts of lyophilized powder of A. camphorata concentrate (D4) and ethyl acetate extracts of A. camphorata mycelium (D5) contained the ingredients that have the potential to be developed into drugs for cancer stem cells. At the same time, the examination of the cell activity tests and IC₅₀ tests of A. camphorata extracts for normal lung fibroblast were conducted. It is worth noting that the half maximal inhibitory concentration (IC₅₀) of ethyl acetate extracts of lyophilized powder of A. camphorata concentrate (D4) and ethyl acetate extracts of A. camphorata mycelium (D5) for normal lung fibroblast were higher than that for cancer stem cells. These results showed that normal lung fibroblast had higher tolerance for A. camphorata extracts (D4 and D5) (Table 1).

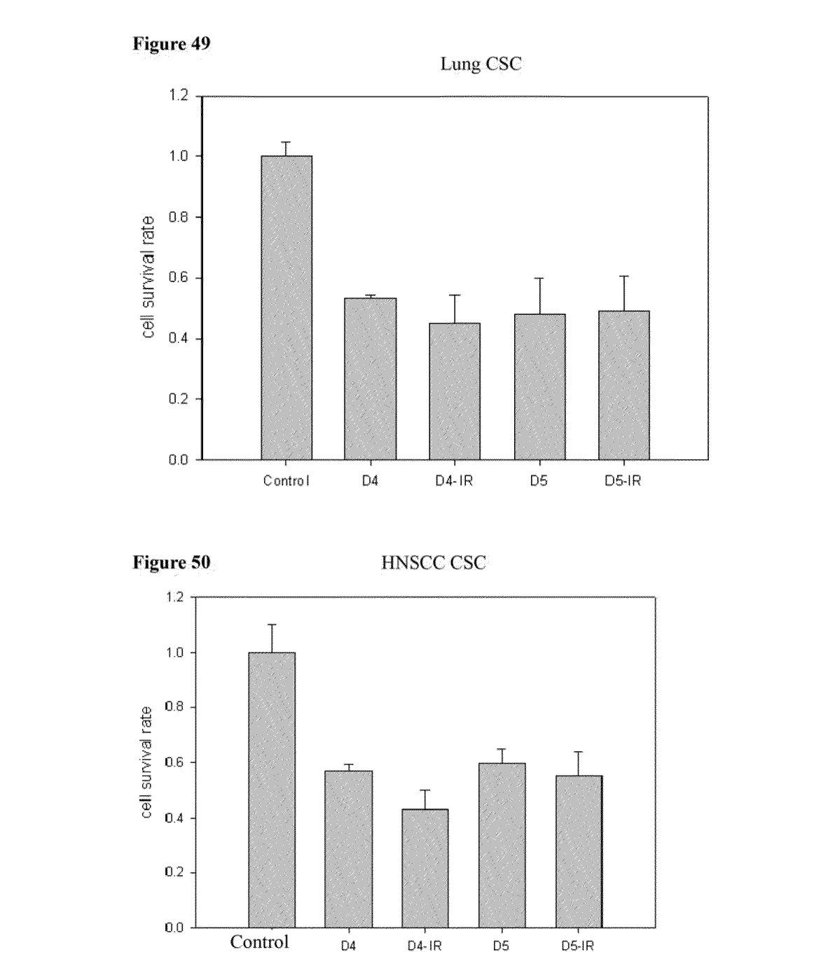

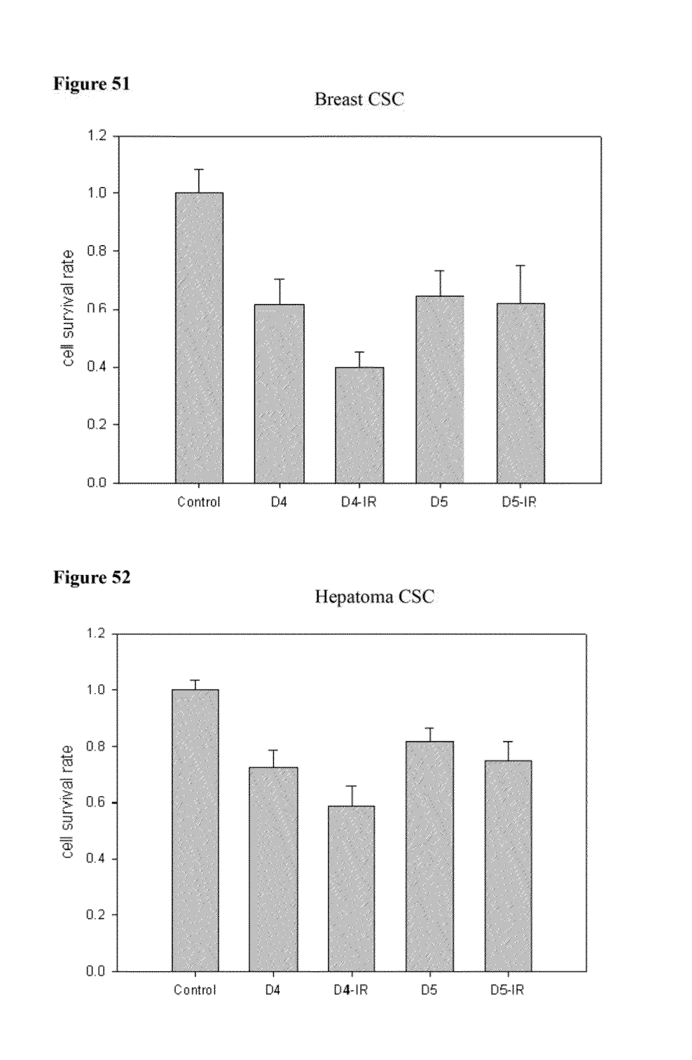

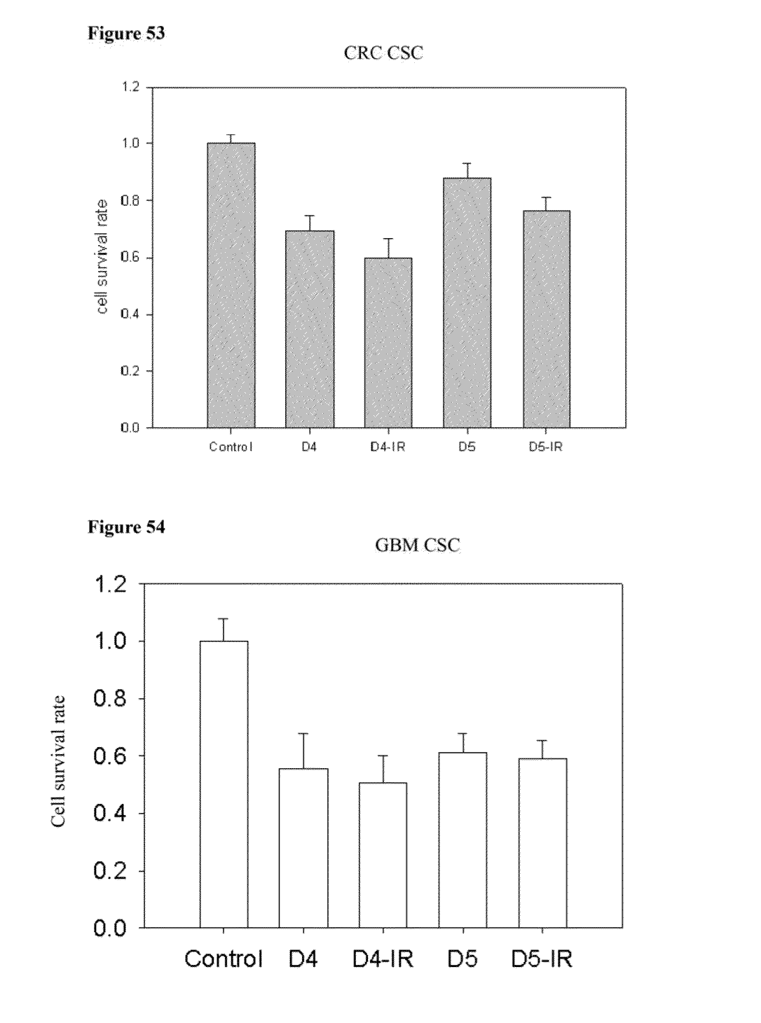

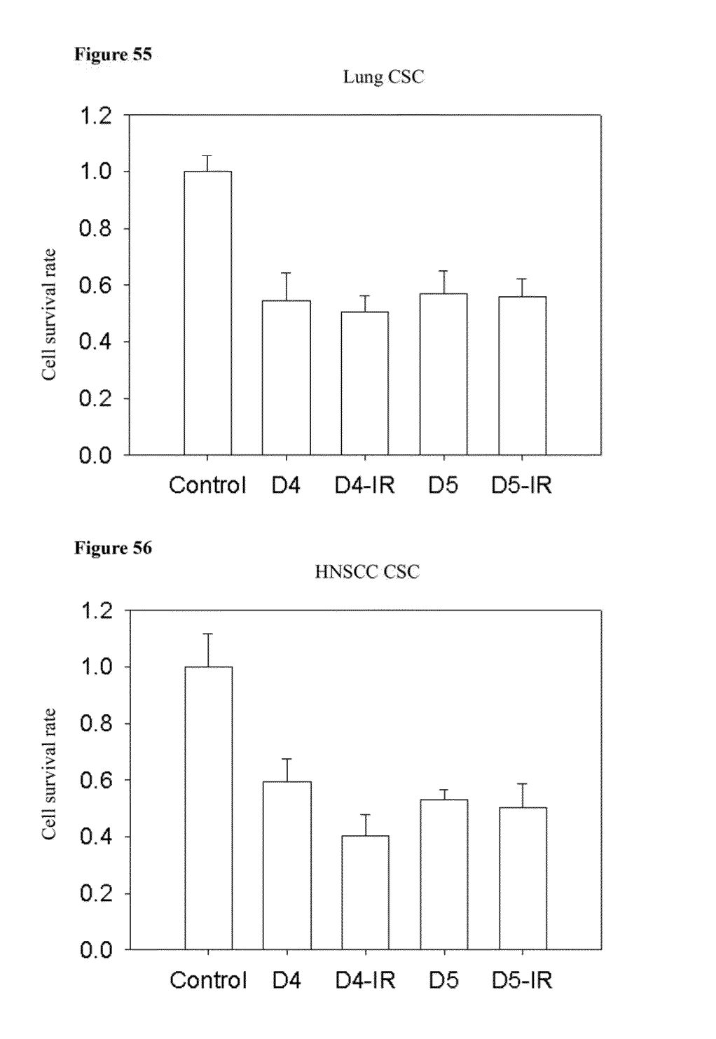

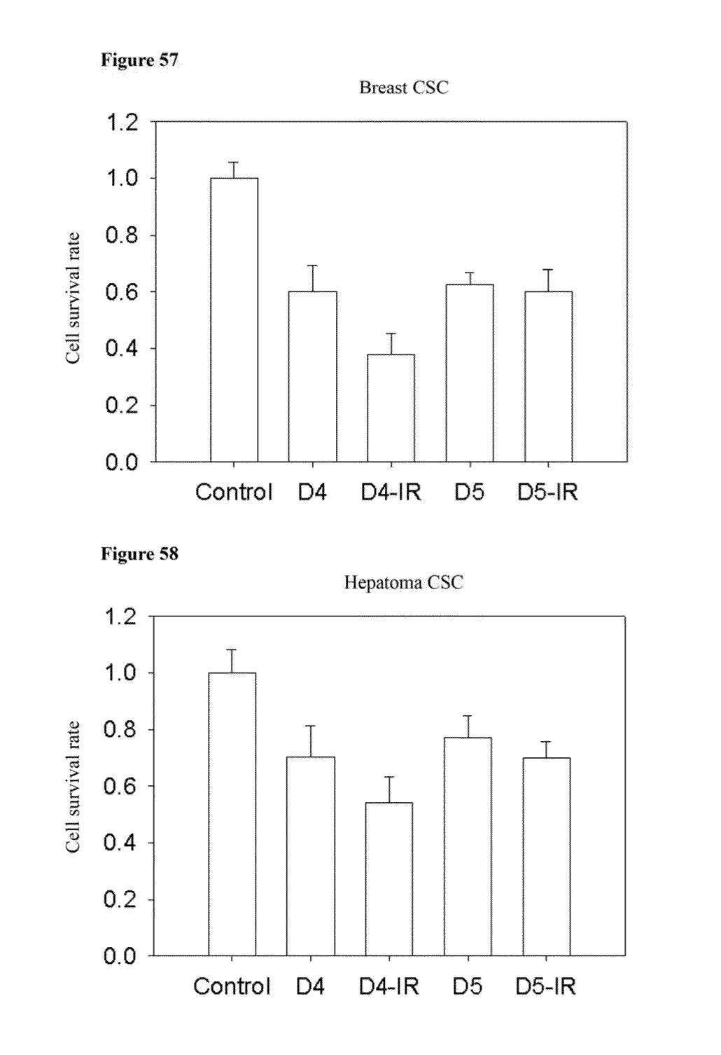

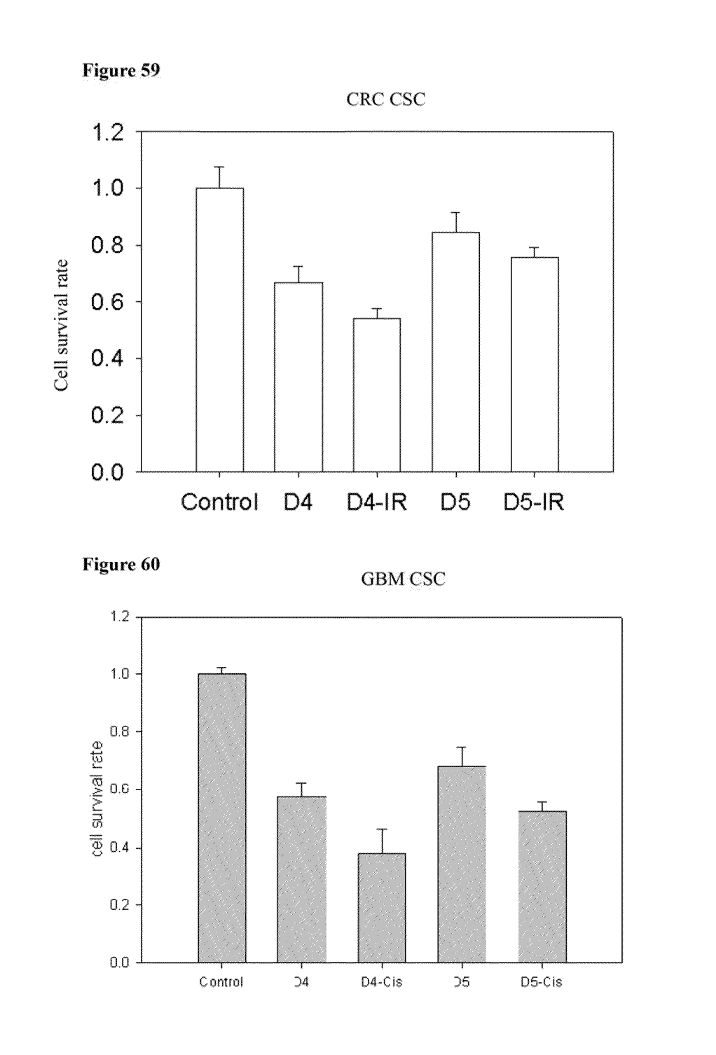

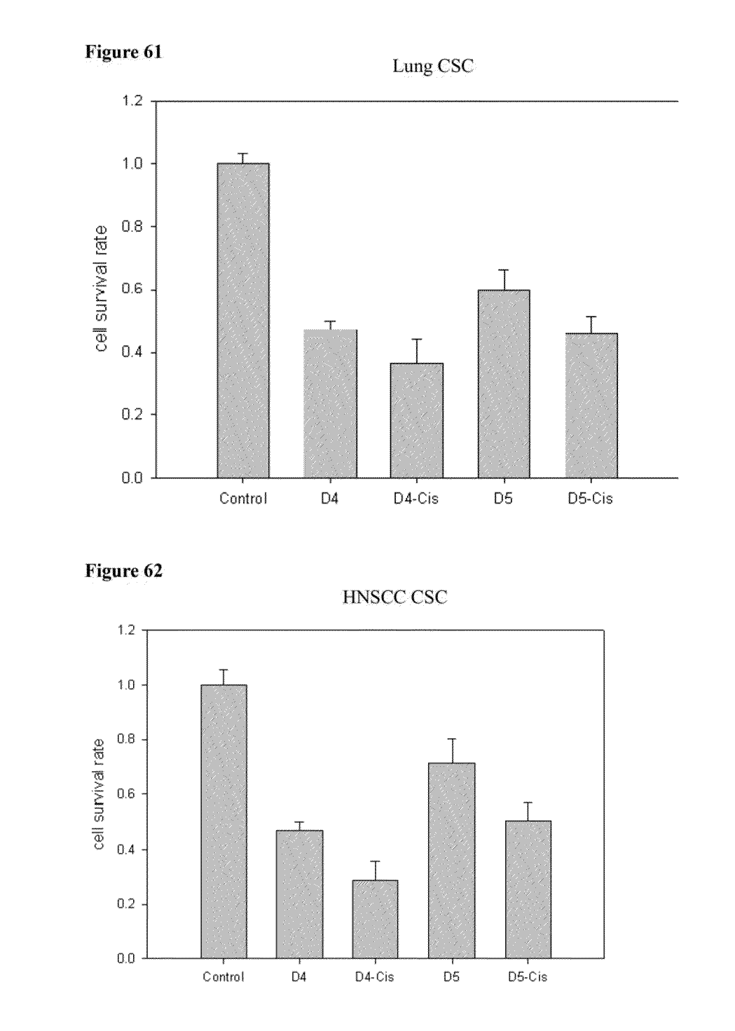

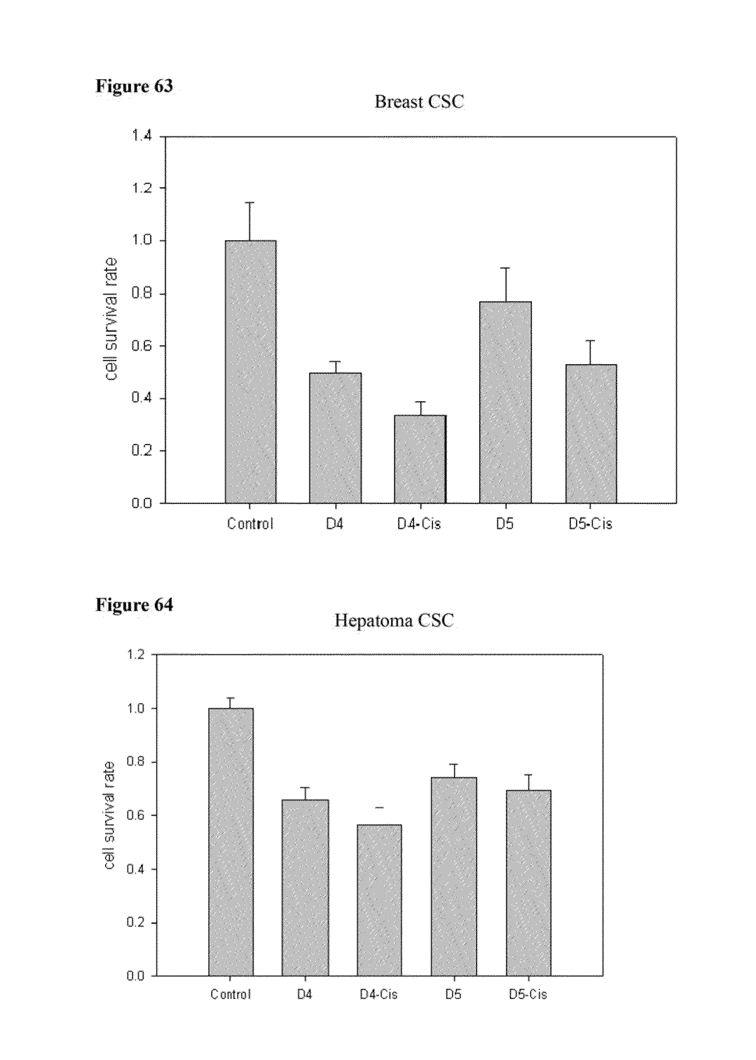

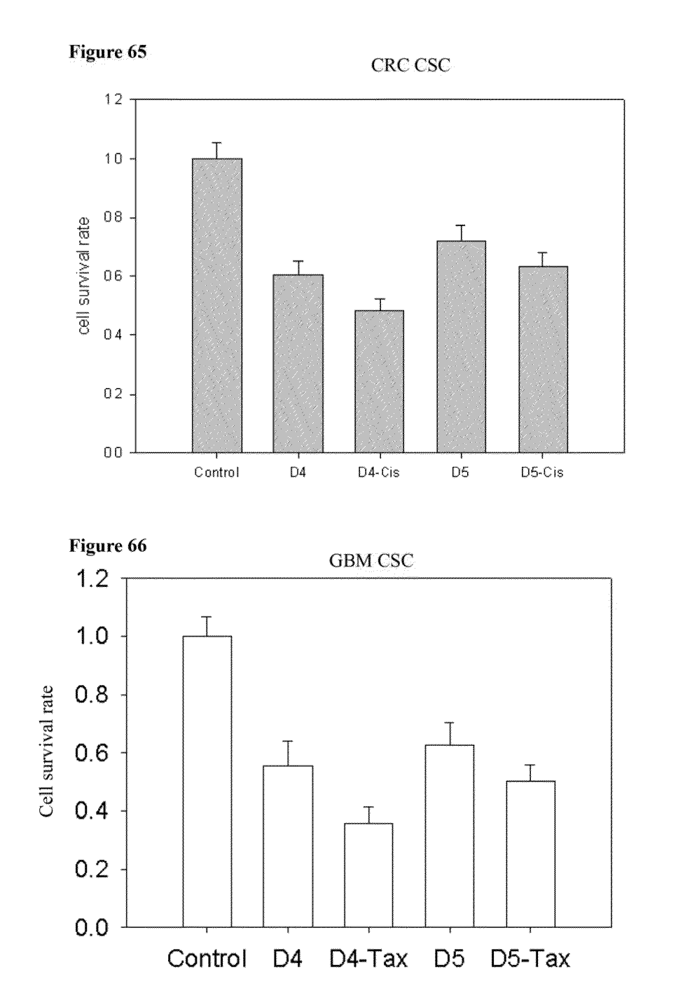

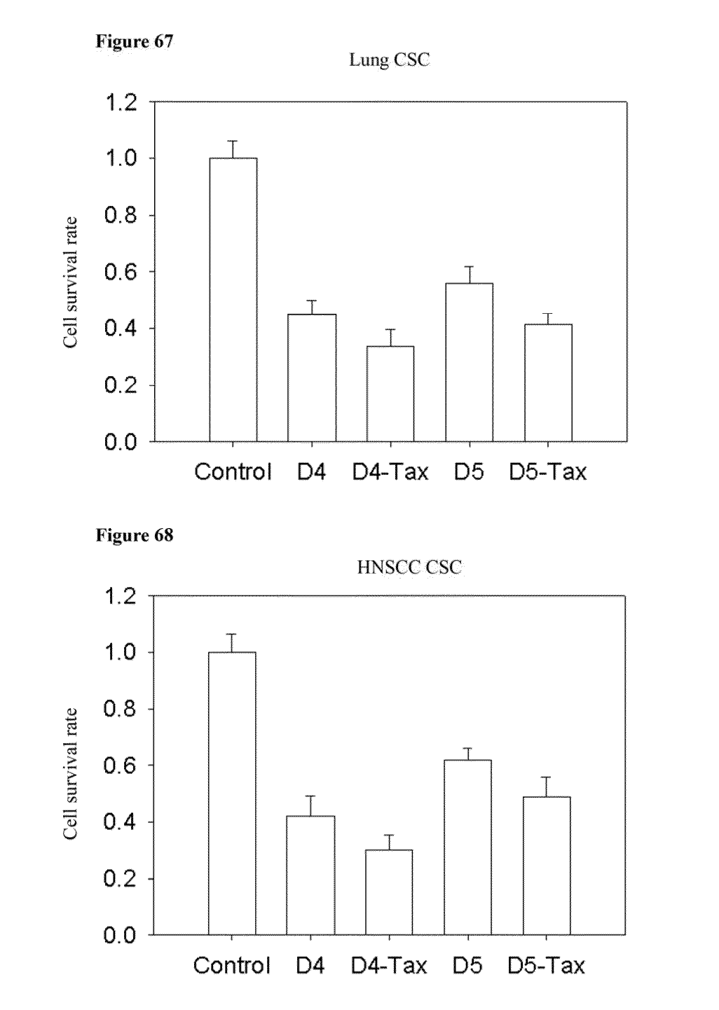

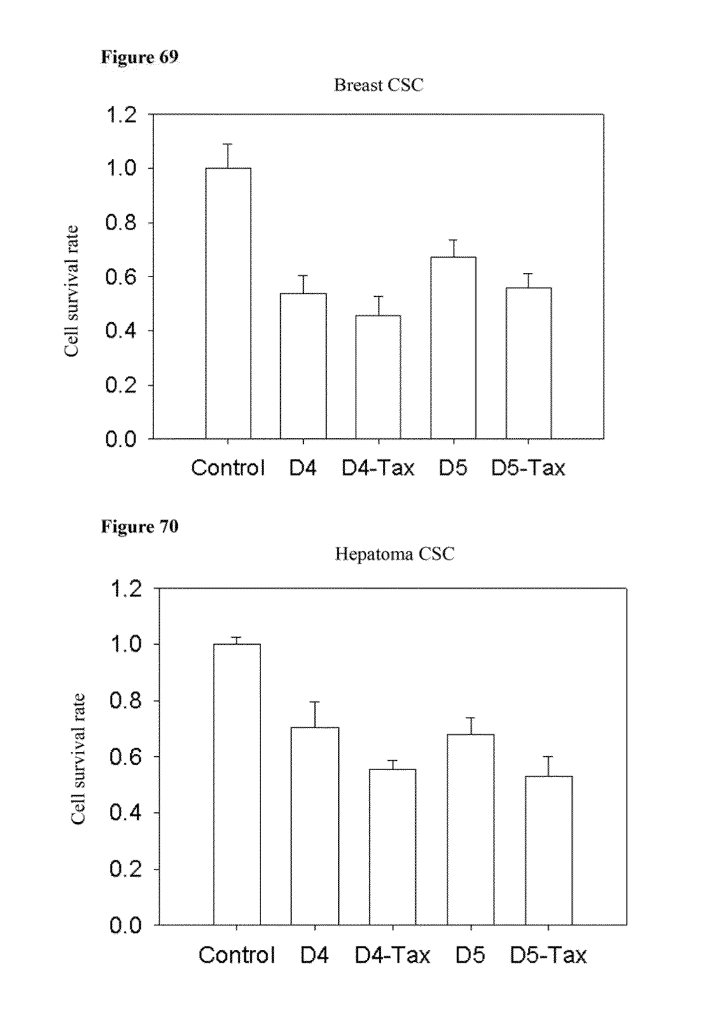

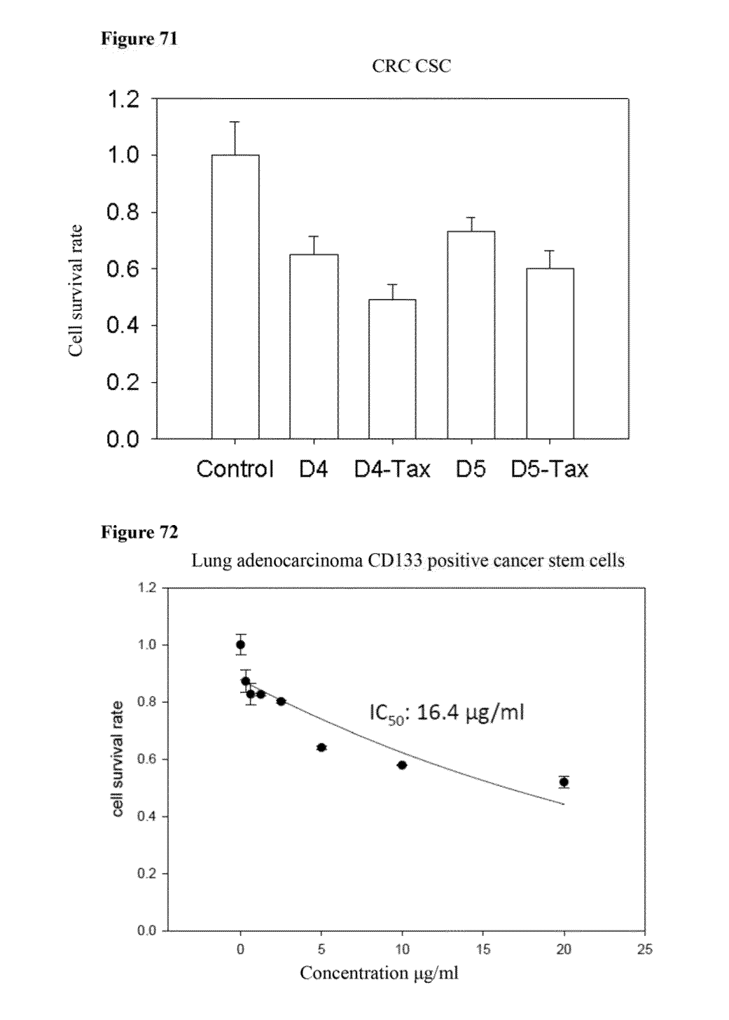

In this embodiment, ethyl acetate extracts of lyophilized powder of A. camphorata concentrate (D4) and ethyl acetate extracts of A. camphorata mycelium (D5) were further tested. Co-administration of the effective A. camphorata extracts with chemotherapy drugs (Cisplatin and Taxol) and co-administration of the effective A. camphorata extracts with ionizing radiation (IR) were tested for anti-cancer effects. The results showed that co-administration of ethyl acetate extracts of lyophilized powder of A. camphorata concentrate (D4) with chemotherapy drugs, Cisplatin, (Table 2 and FIGS. 60-65) or chemotherapy drugs, Taxol, (Table 3 and FIGS. 66-71) partially increased the inhibitory effects on cancer stem cells. Besides, co-administration of ionizing radiation (dosage: 2 Gy or 4 Gy) with ethyl acetate extracts of lyophilized powder of A. camphorata concentrate (D4) partially increased the inhibitory effects on cancer stem cells, D4 had significant inhibitory effects especially with the dosage of 4 Gy of ionizing radiation, (Table 4-6, FIGS. 48-59).

TABLE 2
The half maximal inhibitory concentration (IC₅₀) of
co-administration of A. camphorata extracts with chemotherapy
drugs (Cisplatin: 10 μg/ml) for lung cancer stem cells,
Glioblastomas multiform cancer stem cells, Head and neck
squamous cell carcinoma cancer stem cells, colorectal cancer
stem cells, breast cancer stem cells and Hepatoma cancer
stem cells.

| | D1 | D2 | D3 | D4 | D5 | Cisplatin IC₅₀ |
|---|---|---|---|---|---|---|
| lung | x | x | x | 41.24 μg/ml | 62.17 μg/ml | 17.98 μg/ml |
| cancer | | | | | | |
| stem cells | | | | | | |
| Glioblast- | x | x | x | 52.75 μg/ml | 160.35 μg/ml | 23.33 μg/ml |
| omas | | | | | | |
| multiform | | | | | | |
| cancer | | | | | | |
| stem cells | | | | | | |
| Head and | x | x | x | 38.34 μg/ml | 75.89 μg/ml | 18.87 μg/ml |
| neck | | | | | | |
| squamous | | | | | | |
| cell | | | | | | |
| carcinoma | | | | | | |
| cancer | | | | | | |
| stem cells | | | | | | |
| colorectal | x | x | x | 105.34 μg/ml | 124.77 μg/ml | 42.83 μg/ml |
| cancer | | | | | | |
| stem cells | | | | | | |
| breast | x | x | x | 40.05 μg/ml | 99.43 μg/ml | 20.23 μg/ml |
| cancer | | | | | | |
| stem cells | | | | | | |
| Hepatoma | x | x | x | 106.72 μg/ml | 145.32 μg/ml | 40.15 μg/ml |
| cancer | | | | | | |
| stem cells | | | | | | |

“x”: The maximum drug concentration 400 μg/ml still can not effectively inhibit more than 50% of the cells

TABLE 3
The half maximal inhibitory concentration (IC₅₀) of
co-administration of A. camphorata extracts with chemotherapy
drugs (Taxol: 5 ng/ml) for lung cancer stem cells, Glioblastomas
multiform cancer stem cells, Head and neck squamous cell
carcinoma cancer stem cells, colorectal cancer stem cells, breast
cancer stem cells and Hepatoma cancer stem cells.

| | | | | | | Taxol |
|---|---|---|---|---|---|---|
| | D1 | D2 | D3 | D4 | D5 | IC₅₀ |
| lung cancer | x | x | x | 30.14 μg/ml | 50.23 μg/ml | 13.68 ng/ml |
| stem cells | | | | | | |
| Glioblastomas | x | x | x | 43.67 μg/ml | 105.11 μg/ml | 20.71 ng/ml |
| multiform | | | | | | |
| cancer stem | | | | | | |
| cells | | | | | | |
| Head and neck | x | x | x | 29.76 μg/ml | 60.76 μg/ml | 10.22 ng/ml |
| squamous cell | | | | | | |
| carcinoma | | | | | | |
| cancer stem | | | | | | |
| cells | | | | | | |
| colorectal | x | x | x | 88.15 μg/ml | 107.34 μg/ml | 31.71 ng/ml |
| cancer stem | | | | | | |
| cells | | | | | | |
| breast cancer | x | x | x | 32.42 μg/ml | 88.64 μg/ml | 9.83 ng/ml |
| stem cells | | | | | | |
| Hepatoma | x | x | x | 91.33 μg/ml | 114.23 μg/ml | 33.45 ng/ml |
| cancer stem | | | | | | |
| cells | | | | | | |

“x”: The maximum drug concentration 400 μg/ml still can not effectively inhibit more than 50% of the cells

TABLE 4
The half maximal inhibitory concentration (IC₅₀) of
co-administration of A. camphorata extracts with Ionizing
Radiation (2 Gy) for lung cancer stem cells, Glioblastomas
multiform cancer stem cells, Head and neck squamous cell
carcinoma cancer stem cells, colorectal cancer stem cells,
breast cancer stem cells and Hepatoma cancer stem cells.

| | D1 | D2 | D3 | D4 | D5 |
|---|---|---|---|---|---|
| lung cancer stem cells | x | x | x | 51.23 μg/ml | 68.35 μg/ml |
| Glioblastomas multiform | x | x | x | 60.73 μg/ml | 204.45 μg/ml |
| cancer stem cells | | | | | |
| Head and neck squamous | x | x | x | 40.12 μg/ml | 73.21 μg/ml |
| cell carcinoma cancer stem | | | | | |
| cells | | | | | |
| colorectal cancer stem cells | x | x | x | 122.67 μg/ml | 150.93 μg/ml |
| breast cancer stem cells | x | x | x | 43.19 μg/ml | 110.52 μg/ml |
| Hepatoma cancer stem cells | x | x | x | 137.81 μg/ml | 196.52 μg/ml |

“x”: The maximum drug concentration 400 μg/ml still can not effectively inhibit more than 50% of the cells

TABLE 5
The half maximal inhibitory concentration (IC₅₀) of
co-administration of A. camphorata extracts with
ionizing radiation (4 Gy) for lung cancer stem cells,
Glioblastomas multiform cancer stem cells, Head and
neck squamous cell carcinoma cancer stem cells,
colorectal cancer stem cells, breast cancer stem cells
and Hepatoma cancer stem cells.

| | D1 | D2 | D3 | D4 | D5 |
|---|---|---|---|---|---|
| lung cancer stem cells | x | x | x | 47.51 μg/ml | 60.12 μg/ml |
| Glioblastomas multiform | x | x | x | 50.21 μg/ml | 196.34 μg/ml |
| cancer stem cells | | | | | |
| Head and neck squamous | x | x | x | 42.35 μg/ml | 59.68 μg/ml |
| cell carcinoma cancer stem | | | | | |
| cells | | | | | |
| colorectal cancer stem cells | x | x | x | 101.45 μg/ml | 124.13 μg/ml |
| breast cancer stem cells | x | x | x | 42.69 μg/ml | 92.47 μg/ml |
| Hepatoma cancer stem cells | x | x | x | 110.85 μg/ml | 142.63 μg/ml |

“x”: The maximum drug concentration 400 μg/ml still can not effectively inhibit more than 50% of the cells

TABLE 6
The half maximal inhibitory concentration (IC₅₀) of the
co-administration of A. camphorata extracts with ionizing
radiation (0-4 Gy) for lung cancer stem cells, Glioblastomas
multiform cancer stem cells, Head and neck squamous cell
carcinoma cancer stem cells, colorectal cancer stem cells,
breast cancer stem cells and Hepatoma cancer stem cells.

| | D4 | D4 | D4 | D5 | D5 | D5 |
|---|---|---|---|---|---|---|
| | (0 Gy) | (2 Gy) | (4 Gy) | (0 Gy) | (2 Gy) | (4 Gy) |
| lung cancer | 79.7 | 51.23 | 47.51 | 86.9 | 68.35 | 60.12 |
| stem cells | | | | | | |
| Glioblastomas | 85.5 | 60.73 | 50.21 | 287.8 | 204.45 | 196.34 |
| multiform | | | | | | |
| cancer stem | | | | | | |
| cells | | | | | | |
| Head and neck | 50.9 | 40.12 | 42.35 | 97.7 | 73.21 | 59.68 |
| squamous cell | | | | | | |
| carcinoma | | | | | | |
| cancer stem | | | | | | |
| cells | | | | | | |
| colorectal | 173.4 | 122.67 | 101.45 | 191.2 | 150.93 | 124.13 |
| cancer stem | | | | | | |
| cells | | | | | | |
| breast cancer | 55.8 | 43.19 | 42.69 | 168.6 | 110.52 | 92.47 |
| stem cells | | | | | | |
| Hepatoma | 167.8 | 137.81 | 110.85 | 268.7 | 196.52 | 142.63 |
| cancer stem | | | | | | |
| cells | | | | | | |

unit: μg/ml

The Inhibitory Effects of 4-acetyl-antroquinonol B against the Growth of Tumor Cells

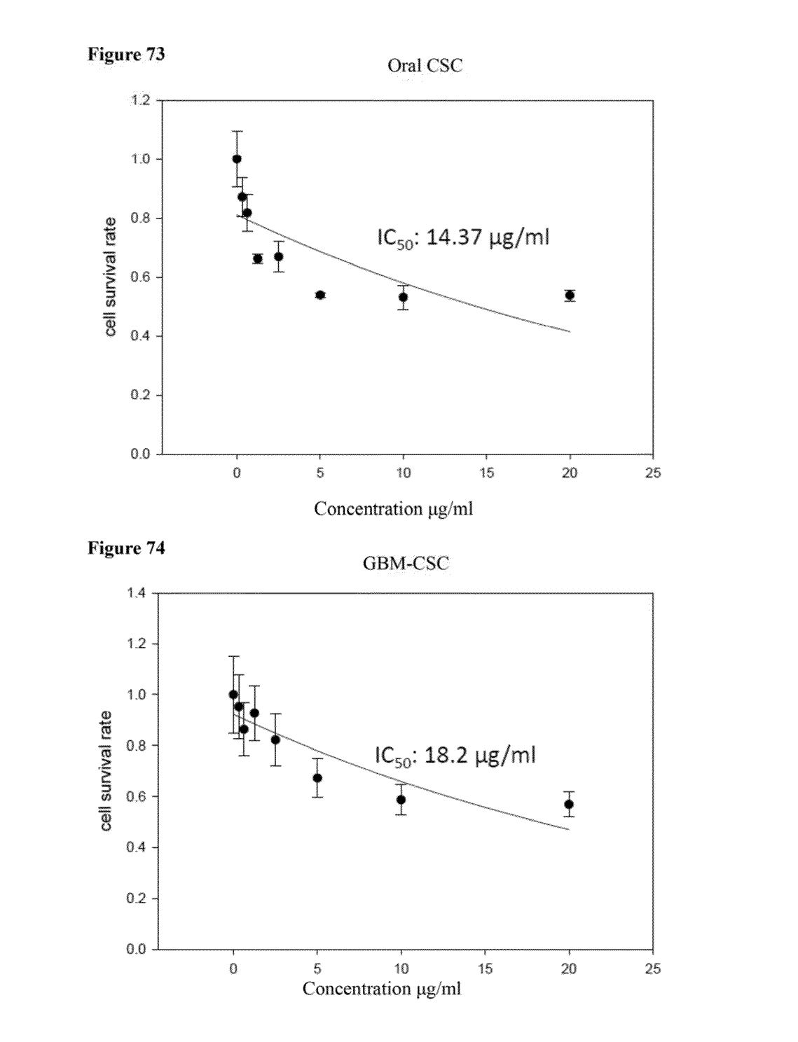

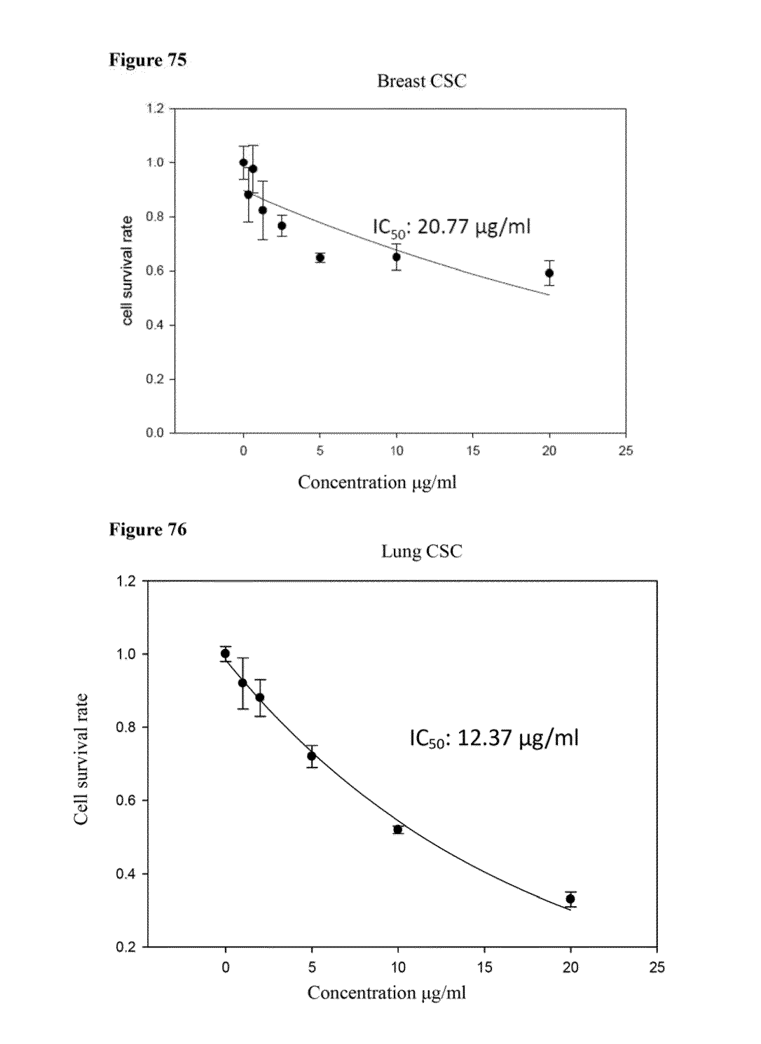

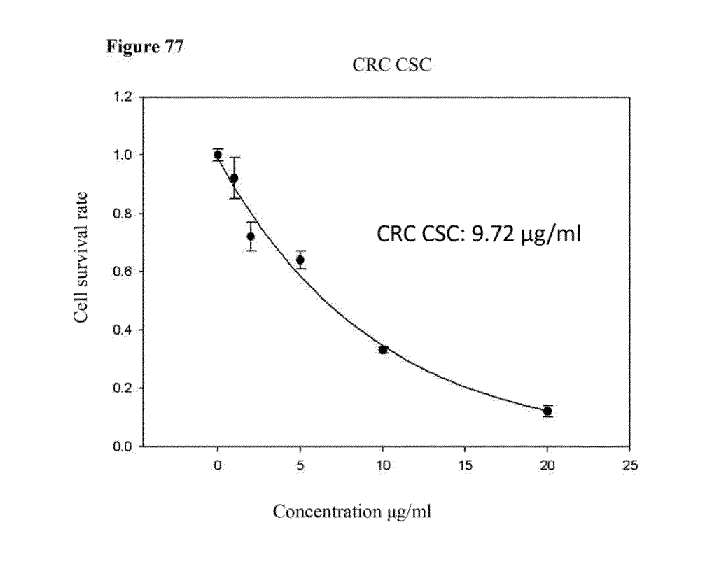

To assess the inhibitory effects of 4-acetyl-antroquinonol B against the growth of cancer stem cells of lung adenocarcinoma CD133 positive tumors, lung cancer, oral cancer, Glioblastomas multiform cancer, breast cancer and colorectal cancer, the cytotoxicity tests for 4-acetyl-antroquinonol B were conducted by MTT assay and they all had effective inhibitory effects on the growth of cancer stem cells, as shown in Table 7 and FIGS. 72-77.

TABLE 7
The half maximal inhibitory concentration (IC50) of
4-acetyl-antroquinonol B for lung adenocarcinoma
CD133 positive cancer stem cells, oral cancer stem
cells, Glioblastomas multiform cancer stem cells, breast
cancer stem cells, lung cancer stem cells and colorectal
cancer stem cells.

| | 4-acetyl-antroquinonol B |
|---|---|
| Lung adenocarcinoma CD133 positive | 16.4 μg/ml |
| cancer stem cells | |
| oral cancer stem cells | 14.37 μg/ml |
| Glioblastomas multiform cancer stem cells | 18.2 μg/ml |
| breast cancer stem cells | 20.77 μg/ml |
| lung cancer stem cells | 12.37 μg/ml |
| colorectal cancer stem cells | 9.72 μg/ml |

What is claimed is:

1. A method for treating cancer caused by cancer stem cells in a subject in need thereof, which comprises administering to the subject an effective amount of Antrodia camphorata extracts.

2. The method of claim 1, wherein the Antrodia camphorata extracts are selected from the group consisting of ethyl acetate extracts of lyophilized powder of Antrodia camphorata concentrate and ethyl acetate extracts of Antrodia camphorata mycelium.

3. The method of claim 1, wherein the cancer stem cells are selected from liver cancer stem cells, lung cancer stem cells, brain tumor stem cells, head and neck cancer stem cells, colorectal cancer stem cells, breast cancer stem cells, leukemia cancer stem cells or gastric cancer stem cells.

4. The method of claim 1, wherein the effective amount ranges from 10 μg/ml to 500 μg/ml.

5. The method of claim 1, which further comprises co-administration of a chemotherapy drug to increase inhibitory effect of the cancer stem cells.

6. The method of claim 5, wherein the chemotherapy drug is selected from Cisplatin or Taxol.

7. The method of claim 1, which further comprises co-administration of an ionizing radiation to increase inhibitory effect of the cancer stem cells.

8. A method for treating cancer caused by cancer stem cells in a subject in need thereof, which comprises administering to the subject an effective amount of 4-acetyl-antroquinonol B.

9. The method of claim 8, wherein the cancer stem cells are selected from lung adenocarcinoma CD133 positive cancer stem cells, lung cancer stem cells, brain tumor stem cells, breast cancer stem cells, oral cancer stem cells or colorectal cancer stem cells.

10. The method of claim 8, wherein the effective amount ranges from 0.1 μg/ml to 100 μg/ml.
